(19) United States
(12) Patent Application Publication
(10) Pub. No.: US 2016/0128933 A1
(43) Pub. Date: May 12, 2016

(54) Therapeutic Methods and Compositions

(71) Applicants: Tiangang Shang, Lexington, MA (US); Guoqing Yu, Tianjin (CN)

(72) Inventors: Tiangang Shang, Lexington, MA (US); Guoqing Yu, Tianjin (CN)

(21) Appl. No.: 14/536,709

(22) Filed: Nov. 10, 2014

(51) Int. Cl. A61K 9/00; A61K 9/70; A61K 31/125; A61K 31/045; A61K 31/35; A61K 31/122; A61K 31/11; A61K 31/194; A61K 31/365; A61K 31/69; A61K 31/05; A61K 31/015; A61K 31/085; A61K 31/12

(52) U.S. Cl. CPC A61K 9/0014; A61K 31/085; A61K 9/7023; A61K 31/125; A61K 31/045; A61K 31/35; A61K 31/12; A61K 31/11; A61K 31/194; A61K 31/365; A61K 31/69; A61K 31/05; A61K 31/015; A61K 31/122

(57) ABSTRACT

Methods and compositions are described for stimulating therapeutic points of a mammal subject by applying therapeutically effective amount of one or more TRP channel agonists.

### FIELD OF THE INVENTION

The present invention relates to a method of stimulating therapeutic points of a mammal subject using agonists of transient receptor potential channels, a method of treating a variety of medical conditions, and compositions comprising of selective agonists of transient receptor potential channels.

### BACKGROUND OF THE INVENTION

Acupuncture and moxibustion have been practiced for thousands of years in China. The practitioners use acupuncture and moxibustion treat or prevent health problems (National Center for Complementary and Alternative Medicine (NCCAM), Traditional Chinese Medicine: An Introduction [online]). A practitioner of acupuncture typically stimulates an acupoint through manual manipulation of a fine needle inserted subcutaneously at an acupoint. A practitioner of moxibustion stimulates an acupoint by burning Artemisia or other herbal materials above the skin to apply heat to the acupoint (National Center for Complementary and Alternative Medicine (NCCAM), Traditional Chinese Medicine: An Introduction [online]). According to traditional Chinese medicine, stimulating acupoints can exert influences upon the body organs. Particularly, by stimulating pre-selected acupoints, the function(s) of a specific body organ can be modulated. Both acupuncture and moxibustion have been used in a variety of medical conditions by traditional Chinese physicians.

The use of both acupuncture and moxibustion has spread to the United States and Europe. Acupuncture has received much more attention than moxibustion has done. According to National Center for Complementary and Alternative Medicine (NCCAM), U.S. National Institute of Health (NIH), millions of Americans use acupuncture each year (National Center for Complementary and Alternative Medicine (NCCAM), Acupuncture [online]). There have been extensive studies conducted on acupuncture. The effects of acupuncture stimulation on the nervous system have been extensively investigated experimentally. Well-documented and reproducible effects on the peripheral, central and autonomic nervous systems have been demonstrated for both manual and electrical acupuncture stimulation in humans and animals (see Napadow, V. et al. The Status and Future of Acupuncture Mechanism Research, J. Altern. Complement Med., Vol. 14, no. 7(2008), pp. 861-869). It has been found that many, if not all, of acupoints are located over or in the close proximity of major somatic neural tracts (see Longhurst, J. C. “Acupuncture.” In: Robertson, D., Primer on The Autonomic Nervous System (California, San Diego, 2004), pp. 426-429) and richly innervated by peripheral nerves (see Zhao, Z. Q. Neural mechanism underlying acupuncture analgesia. Prog. Neurobiol., Vol. 85, no. 4 (2008), pp. 355-375). The action potentials can be induced by stimulating the receptors underneath the acupoints using acupuncture needles, pressure, or heat. Stimulated action potentials are transmitted by afferent nerve fibers to the central nervous system. See, for example, Longhurst, J. C. “Acupuncture.” In: Robertson, D., Primer on The Autonomic Nervous System (California, San Diego, 2004), pp. 426-429. The responses of central nervous system to the stimulations at the acupoints have been experimentally demonstrated by using functional magnetic resonance imaging (fMRI). A study conducted by Hui et al. (see Hui, K. K. et al. Acupuncture modulates the limbic system and subcortical gray structures of the human brain: Evidence from fMRI studies in normal subjects. Hum. Brain Mapp., Vol. 9, no. 1 (2000), pp. 13-25), demonstrated that acupuncture needle manipulation modulated the activity of the limbic system and subcortical structures. A study by Siedentopf et al. (see Siedentopf, C. M. et al. Functional magnetic resonance imaging detects activation of the visual association cortex during laser acupuncture of the foot in humans. Neurosci. Lett., Vol. 327, no. 1 (2002), pp. 53-56), showed that laser acupuncture at a specific acupoint led to activation of visual brain areas. Studies also showed that the responses of central nervous system to acupoint stimulations were different from those to non-acupoint stimulations. A study by Napadow et al. (see Napadow, V. et al. Effects of electroacupuncture versus manual acupuncture on the human brain as measured by fMRI. Hum. Brain Mapp., Vol. 24, no. 3 (2005), pp. 193-205), demonstrated that the stimulations at acupoint ST36 (Zusanli) produced more widespread responses than non-acupoint stimulations. Specifically, acupuncture stimulations produced hemodynamic signal increase in the anterior insula and decrease in limbic and paralimbic structures including the amygdala, anterior hippocampus and the cortices of the subgenual and retrosplenial cingulate, ventromedial prefrontal cortex, frontal and temporal poles, while these results were not observed for non-acupoint stimulations. Another study, by Na et al. (see Na, B. et al. An fMRI study of neuronal specificity of an acupoint: Electroacupuncture stimulation of Yanglingquan (GB34) and its sham point. Neurosci. Lett., Vol. 464 (2009), pp. 1-5), investigated the neuronal specificity of acupoint Yanglingquan (GB34) with electroacupuncture stimulation using functional magnetic resonance imaging (fMRI). Na et al. reported that the electroacupuncture stimulations at the left GB34 specifically activated the right putamen, caudate body, claustrum, thalamus, cerebellum, as well as the left caudate body, ventral lateral thalamus and cerebellum, whereas the electroacupuncture stimulations at the non-acupoint (i.e., the point 2.0 cm lateral to GB34) specifically activated the right BA6, BA8, BA40, BA44, thalamus, as well as the left thalamus and cerebellum. Na et al. concluded that these findings supported neuronal specificity of the acupoint studied. Furthermore, many studies have demonstrated that acupoint stimulations can produce therapeutic action. A study by Longhurst et al. (see Longhurst, J. C. et al. Reversal of reflex-induced myocardial ischemia by median nerve stimulation: a feline model of electroacupuncture. Circulation, Vol. 97 (1998), pp. 1186-1194), examined the effects of electroacupuncture on cardiac ischemia. Longhurst et al. developed a feline model of demand-induced myocardial ischemia, in which the left anterior descending coronary artery was ligated, partially allowing normal coronary blood flow at rest but causing an insufficient flow and hence transient ischemia, identified by the regional myocardial dysfunction, during reflex stimulation evoked by applying bradykinin to gallbladder. Longhurst et al. reported that electroacupuncture stimulations at acupoint PC6 (Neiguan) significantly improved regional myocardial ischemia induced by restriction of coronary blood flow and superimposed stimulation of the cardiovascular sympathetic nervous system. A later study by Diego et al. (see Diego, M. M. et al. Short-term effects of acupuncture on pulmonary function and heart rate in patients with chronic obstructive pulmonary diseases. J. Chin. Med., Vol. 94 (2010), pp. 18-19), reported that manual acupuncture stimulations could improve the respiratory function of patients with chronic obstructive pulmonary disease. In this study, six individuals with a diagnosis of chronic obstructive pulmonary disease were assessed before and after the stimulations at the acupoints GV14 (Dazhui), CV22 (Tiantu), BL13 (Feishu), BL23 (Shenshu) and LU10 (Yuji). Diego et al. reported that a significant increase in forced vital capacity and decrease in heart rate after treatment were observed. A retrospective study by Lu et al. (see Lu, K. et al. A retrospective review of acupuncture use for the treatment of pain in sickle cell disease patients: descriptive analysis from a single institution. Clin. J. Pain, Vol. 30, no. 9 (2014), pp. 825-830), showed that the use of acupuncture could be beneficial for adult sickle cell patients' pain management.

Overall, these studies demonstrated that action potentials produced at acupoints can be transmitted by afferent nerve fibers to the central nervous system and cause the responses in a number of regions in the central nervous system, which lead to the modulation of visceral functions and producing therapeutic action. Similarly, stimulations at trigger points, “ah shi” points and motor points can also produce therapeutic action (see for example, Vulfsons, S. et al. Trigger point needling: techniques and outcome. Curr. Pain Headache Rep., Vol. 16, no. 5 (2012), pp. 407-412; Gunn, C. C. et al. Dry needling of muscle motor points for chronic low-back pain: a randomized clinical trial with long-term follow-up. Spine, Vol. 5, no. 3 (1980), pp. 279-291; Son, B. K. et al. Efficacy of ah shi point acupuncture on acne vulgaris. Acupunct. Med., Vol. 28, no. 3 (2010), pp. 126-129). “Ah shi” points are local points of tenderness and often used in acupuncture independently or in conjunction with acupoints. A trigger point is a tender spot in a muscle often with a palpable taut band of tissue that elicits pain referral when pressure is applied (see Rock, J. M. et al. Treatment of nonspecific thoracic spine pain with trigger point dry needling and intramuscular electrical stimulation: a case series. Int. J. Sports Phys. Ther., Vol. 9, no. 5 (2014), pp. 699-711). A motor point is a site of skin area above the muscle in which an electrical pulse applied transcutaneously evokes a muscle twitch with the least injected current (see Gobbo, M. et al. Muscle motor point identification is essential for optimizing neuromuscular electrical stimulation use. J. Neuroeng. Rehabil. 2014 11:17). Throughout the present specification, a “therapeutic point” can be an acupoint, “ah shi” point, trigger point and motor point.

A few methods have been utilized in traditional Chinese medicine to initiate action potentials at therapeutic points. Among them, acupuncture initiates action potentials by inserting a fine needle into or beneath the skin and leaving the needle in place or sometimes manipulating the needle manually, while moxibustion induces action potentials by applying heat to therapeutic points. Electroacupuncture is a relatively new acupoint stimulation method. It applies electric stimulations to therapeutic points. Electroacupuncture involves inserting a needle into a therapeutic point and attaching an electrode to the needle to provide a pulsating electric current (see Institute for Traditional Medicine, Electro-Acupuncture [online]). It is difficult for an ordinary person to conduct acupuncture and electroacupuncture. A licensed acupuncturist is required to perform acupuncture and electroacupuncture. There are risks associated with the acupuncture and electroacupuncture practice by people who lack the education and supervised clinical training. The risks include blood vessel, nerve and organ injury from inappropriate acupuncture needle angle and depth of insertion or from inappropriate acupuncture needle manipulation (see Wisconsin Society of Certified Acupuncturists (WISCA), The Illegal and Unsafe Practice of Acupuncture under the Term “Dry Needling:” 10 Facts You Should Know [online]). Particularly, electroacupuncture should be used cautiously in patients suffering from serious heart diseases. It is not recommended to place the electrodes near the heart because it could respond unfavorably to the electrical pulses. Besides, the path between two electrodes must not cross the area of the heart in spite of the use of low currents. Some experts suggest that the electrodes attached to the acupuncture needles should not be placed on both the sides of spinal cord due to the probable effect of electric stimulus on nervous system (see Electro Acupuncture Machine, Advantages and Disadvantages of Electro Acupuncture Therapy [online]). In addition, acupuncture needles should be appropriately sterilized to prevent patients from suffering infectious disease (see Wisconsin Society of Certified Acupuncturists (WISCA), The Illegal and Unsafe Practice of Acupuncture under the Term “Dry Needling:” 10 Facts You Should Know [online]). Therefore, it is not recommended to perform self-treatment using acupuncture and/or electroacupuncture by an ordinary person who lacks the training. There are also risks associated with moxibustion by people who lack the education and supervised clinical training. Moxibustion involves the burning of herbs. It may cause skin burns if conducted inappropriately. In addition, moxibustion is a thermal treatment method, it is proposed to avoid applying moxibustion to the patients with any kind of symptoms of “heat syndrome” diagnosed according to the criteria of the Eight Principles in traditional Chinese medicine as well as the patients with strong heat signs such as high fever, etc. (see Wellness, Moxibustion Evidence [online]). It is recommended to conduct moxibustion by experienced or qualified practitioners for the sake of safety. A self moxibustion treatment is not recommended. On the other hand, in acupuncture and electroacupuncture, the acupuncture needles typically stay in place for 15-40 minutes. It is not practical in acupuncture and electroacupuncture to maintain sustained therapeutic stimulations over a long duration. Similarly, it is not practical in moxibustion to keep sustained thermal stimulation over a long duration too. In summary, traditional methods for stimulating therapeutic points including acupuncture, electroacupuncture and moxibustion have some limitations. Therefore, a need exists for methods, devices and/or systems that provide non-invasive and sustained therapeutic stimulations over a long duration. Meanwhile, these methods, devices and/or systems should be safe, be easy to use, be economical and allow patients to perform self-treatment under appropriate instructions. Stimulating therapeutic points using active substances is a promising candidate.

Several compositions have been disclosed. These compositions are applied to one or more relevant acupoints to generate an ameliorating or treatment effect. You, in U.S. Patent Application Publication Nos. 20130304167, 20130172970, 20130172969, 20130171231 and 20130171232, describes a composition for ameliorating or treating back pain, obesity, a tumor condition, insomnia and Parkinson's disease, respectively. The composition comprises a heat generation component, as well as several additional components including Saussurea in volucrata, musk, sea otter, Chuanwu, Caowu, lilac, menthol, Meiyao, Wujiapi and Maqianzhi. The composition is formulated as a patch. The heat generation component can generate heat upon exposure to moisture or air that provides a local temperature above body temperature (e.g., 37° C.) to about 50° C. The composition is selectively applied to a set of acupoints or one or more acupoints for back pain, obesity, a tumor condition, insomnia and Parkinson's disease, respectively, to generate an ameliorating or treatment effect. Because the composition generates heat upon application to a set of acupoints or one or more acupoints, caution should be taken to avoid burning injures when using the composition. Meanwhile, the heat generated may prevent some people from using the composition, such as the people with any kind of symptoms of “heat syndrome” diagnosed according to the criteria of the Eight Principles in traditional Chinese medicine as well as the people with strong heat signs such as high fever. Moreover, the composition contains a variety of ingredients. The effective ingredients are not specified. The effective quality control can sometimes be difficult. In addition, some ingredients may cause allergic reactions or other adverse effects, one has to pay attention to make sure that none of ingredients would cause allergic reactions or other adverse reactions before applying the composition. U.S. Pat. No. 7,645,462 to Sun discloses a patch for applying a medication to the acupoints of a patient's body. The medication comprises a mixture of water extracts of Ligusticum, Salvia root, Tangkuei and chrysanthemum. The acupoint path is applied to designated acupoints and the medication enters blood circulation through transdermal absorption and performs a sustainable regulating function. The acupoint patch contains a variety of ingredients. Except ferulic acid in Tangkuei, none of the effective ingredients are specified. Therefore, the effective quality control can sometimes be difficult. In addition, some ingredients may cause allergic reactions or other adverse effects, one has to pay attention to make sure that none of ingredients would cause allergic reactions or other adverse reactions before applying the composition. U.S. Pat. No. 6,193,995 to Zhenzhen et al., describes an herbal mixture for treating nasal congestion. The herbal mixture comprises Fructus Piperis nigri herb, Rhizoma Zingiberis officinalis herb and Ramulus Cinnamomi Cassiae herb as well as an adhesive. The herbal mixture is applied to one or more acupoints on a user's face. The herbal mixture involves various ingredients and the effective ingredients are not specified. The effective quality control can sometimes be difficult. In addition, some ingredients may cause allergic reactions or other adverse effects, one has to pay attention to make sure that none of ingredients would cause allergic reactions or other adverse reactions before using the herbal mixture. U.S. Pat. No. 7,595,066 to Kim discloses a composition with moxibustion effects as well as a press pellet using the composition. The composition comprises 20˜40% by weight of a concentrated Artemisia extract, an auxiliary herbal material, a water-soluble tincture, 30%˜60% by weight a far-infrared radiating powder and an adhesive member. The composition is applied to a set of acupoints or one or more acupoints of a patient's body. Kim explains that a large quantity of energy released by far-infrared powders replaces heat required to burn Artemisia in moxibustion and the use of the far-infrared radiating powders provides the desired moxibustion and acupuncture effects without heat. The composition contains a variety of ingredients. The effective ingredients are not specified. The effective quality control can sometimes be difficult. In addition, some ingredients may cause allergic reactions or other adverse effects, one has to pay attention to make sure that none of ingredients would cause allergic reactions or other adverse reactions before applying the composition. JOO, et al., in U.S. Patent Application Publication No. 2011/0123599, describes an aroma therapy patch exerting aroma therapeutic effects by the actions of particular herbal plants as well as chromatherapeutic effects induced by the wavelength of a specific color. The aroma therapy patch is comprised of an herbal layer containing the herbal extract and an adhesive layer. At least one substrate surface has color on it. The herbal extract is the extract of a herbal plant selected from the group consisting of lavender, rosemary, lemon, jasmine, chamomile, eucalyptus globulus, peppermint, mate, salvia, thyme, yarrow achillea millefolium, ylang-ylang, tea tree, fennel, cinnamon, frank incense, myrrh and a mixture thereof. The herbal layer may further comprises a far-infrared emission material or an anion emission material. The aroma therapy patches are applied to appropriate acupoints of the human body to treat or alleviate diseases, or to improve the body functions of the human body. The composition contains a variety of ingredients. The effective ingredients are not specified. The effective quality control can sometimes be difficult. In addition, some ingredients may cause allergic reactions or other adverse effects, one has to pay attention to make sure that none of ingredients would cause allergic reactions or other adverse reactions before applying the composition.

Accordingly, there is a need for a composition which can be applied to stimulate therapeutic points and whose effective ingredients are specified. The present invention fulfills this need and provides other related advantages.

### SUMMARY OF THE INVENTION

In one aspect, the present invention relates to a method of stimulating therapeutic point(s) of a mammal subject. The method includes applying to a set of therapeutic points or one or more therapeutic points of a mammal subject a therapeutically effective amount of a composition comprising an agonist of a transient receptor potential (TRP) channel selected from the group consisting of TRPV1, TRPV2, TRPV3, TRPV4, TRPA1, TRPM8, and combinations thereof.

The TRP channel agonist applied in the method of stimulating a set of therapeutic points or one or more therapeutic points of a mammal subject can be an agonist of one or more of TRPV1, TRPV2, TRPV3, TRPV4, TRPA1 and TRPM8. One or more TRP channel agonists can be applied. In some embodiments, two or more agonists that activate the same or different TRP channels are applied. In some embodiments, an agonist that activates two or more TRP channels is applied. The method described herein are not limited to any specific agonist. A variety of agonists of TRPV1, TRPV2, TRPV3, TRPV4, TRPA1 and TRPM8 can be used.

In some embodiments, the compositions employed in the method of stimulating a set of therapeutic points or one or more therapeutic points of a mammal subject, may further comprise one or more penetration enhancing agents. In some embodiments, the penetration enhancing agent included in the compositions employed in the method of stimulating one or more therapeutic points of a mammal subject is selected from the group consisting of terpinen-4-ol, α-terpineol and combinations thereof.

In some embodiments, the compositions employed in the method of stimulating a set of therapeutic points or one or more therapeutic points of a mammal subject, may further comprise one or two compounds selected from the group consisting of trans-caryophyllene and thujone.

In some embodiments, the compositions employed in the method of stimulating a set of therapeutic points or one or more therapeutic points of a mammal subject, comprise: an agonist of a TRP channel selected from the group consisting of TRPV1, TRPV2, TRPV3, TRPV4, TRPA1, TRPM8, and combinations thereof; one or more penetration enhancing agents; and one or two compounds selected from the group consisting of trans-caryophyllene and thujone. In some embodiments, the penetration enhancing agent included in the compositions employed in the method of stimulating one or more therapeutic points of a mammal subject is selected from the group consisting of terpinen-4-ol, α-terpineol and combinations thereof.

In some embodiments, the compositions employed in the method of stimulating a set of therapeutic points or one or more therapeutic points of a mammal subject, comprise one or more TRP channel agonists selected from the group consisting of 2-aminoethoxydiphenol borate, borneol, bisandrographolide A, camphor, 1,8-cineole, citral, citrate, eugenol and 6-gingerol.

In some embodiments, the compositions employed in the method of stimulating a set of therapeutic points or one or more therapeutic points of a mammal subject, comprise one or more TRP channel agonists selected from the group consisting of 2-aminoethoxydiphenol borate, borneol, bisandrographolide A, camphor, 1,8-cineole, citral, citrate, eugenol, 6-gingerol and one or more penetration enhancing agents. In some embodiments, the penetration enhancing agent included in the compositions employed in the method of stimulating one or more therapeutic points of a mammal subject is selected from the group consisting of terpinen-4-ol, α-terpineol and combinations thereof.

In some embodiments, the compositions employed in the method of stimulating a set of therapeutic points or one or more therapeutic points of a mammal subject, comprise one or more TRP channel agonists selected from the group consisting of 2-aminoethoxydiphenol borate, borneol, bisandrographolide A, camphor, 1,8-cineole, citral, citrate, eugenol, 6-gingerol, and one or two compounds selected from the group consisting of trans-caryophyllene and thujone.

In some embodiments, the compositions employed in the method of stimulating a set of therapeutic points or one or more therapeutic points of a mammal subject comprise: one or more TRP channel agonists selected from the group consisting of 2-aminoethoxydiphenol borate, borneol, bisandrographolide A, camphor, 1,8-cineole, citral, citrate, eugenol and 6-gingerol; one or more penetration enhancing agents; and one or two compounds selected from the group consisting of trans-caryophyllene and thujone. In some embodiments, the penetration enhancing agent included in the compositions employed in the method of stimulating one or more therapeutic points of a mammal subject is selected from the group consisting of terpinen-4-ol, α-terpineol and combinations thereof.

In some embodiments, the compositions employed in the method of stimulating a set of therapeutic points or one or more therapeutic points of a mammal subject are applied topically as a patch. In some embodiments, the compositions employed in the method of stimulating a set of therapeutic points or one or more therapeutic points of a mammal subject are applied topically in liquid form.

In another aspect of the present invention, it is provided a method of treating a disease of a mammal subject. The method comprises: choosing a set of therapeutic points or one or more therapeutic points of the mammal subject based on subject's symptoms; and then applying to the chosen therapeutic point(s) of the subject a therapeutically effective amount of a composition comprising an agonist of a TRP channel selected from the group consisting of TRPV1, TRPV2, TRPV3, TRPV4, TRPA1, TRPM8, and combinations thereof.

The TRP channel agonist applied in the method of treating a disease of a mammal subject can be an agonist of one or more of TRPV1, TRPV2, TRPV3, TRPV4, TRPA1 and TRPM8. One or more TRP channel agonists can be applied. In some embodiments, two or more agonists that activate the same or different TRP channels are applied. In some embodiments, an agonist that activates two or more TRP channels is applied. The method described herein are not limited to any specific agonist. A variety of agonists of TRPV1, TRPV2, TRPV3, TRPV4, TRPA1 and TRPM8 can be used.

In some embodiments, the compositions employed in the method of treating a disease of a mammal subject, may further comprise one or more penetration enhancing agents. In some embodiments, the penetration enhancing agent included in the compositions employed in the method of treating a disease of a mammal subject is selected from the group consisting of terpinen-4-ol, α-terpineol and combinations thereof.

In some embodiments, the compositions employed in the method of treating a disease of a mammal subject, may further comprise one or two compounds selected from the group consisting of trans-caryophyllene and thujone.

In some embodiments, the compositions employed in the method of treating a disease of a mammal subject comprise an agonist of a TRP channel selected from the group consisting of TRPV1, TRPV2, TRPV3, TRPV4, TRPA1, TRPM8, and combinations thereof; one or more penetration enhancing agents; and one or two compounds selected from the group consisting of trans-caryophyllene and thujone. In some embodiments, the penetration enhancing agent included in compositions employed in the method of treating a disease of a mammal subject is selected from the group consisting of terpinen-4-ol, α-terpineol and combinations thereof.

In some embodiments, the compositions employed in the method of treating a disease of a mammal subject, comprise one or more TRP channel agonists selected from the group consisting of 2-aminoethoxydiphenol borate, borneol, bisandrographolide A, camphor, 1,8-cineole, citral, citrate, eugenol and 6-gingerol.

In some embodiments, the compositions employed in the method of treating a disease of a mammal subject, comprise one or more TRP channel agonists selected from the group consisting of 2-aminoethoxydiphenol borate, borneol, bisandrographolide A, camphor, 1,8-cineole, citral, citrate, eugenol, 6-gingerol and one or more penetration enhancing agents. In some embodiments, the penetration enhancing agent included in the compositions employed in the method of treating a disease of a mammal subject is selected from the group consisting of terpinen-4-ol α-terpineol and combinations thereof.

In some embodiments, the compositions employed in the method of treating a disease of a mammal subject, comprise one or more TRP channel agonists selected from the group consisting of 2-aminoethoxydiphenol borate, borneol, bisandrographolide A, camphor, 1,8-cineole, citral, citrate, eugenol, 6-gingerol, and one or two compounds selected from the group consisting of trans-caryophyllene and thujone.

In some embodiments, the compositions employed in the method of treating a disease of a mammal subject, comprise: one or more TRP channel agonists selected from the group consisting of 2-aminoethoxydiphenol borate, borneol, bisandrographolide A, camphor, 1,8-cineole, citral, citrate, eugenol and 6-gingerol; one or more penetration enhancing agents; and one or two compounds selected from the group consisting of trans-caryophyllene and thujone. In some embodiments, the penetration enhancing agent included in the compositions employed in the method of treating a disease of a mammal subject is selected from the group consisting of terpinen-4-ol, α-terpineol and combinations thereof.

In some embodiments, the compositions employed in the method of treating a disease of a mammal subject are applied topically as a patch. In some embodiments, the compositions employed in the method of treating a disease of a mammal subject are applied topically in liquid form.

In some embodiments, the therapeutic points employed in the methods of treating a disease of a mammal subject include one or more therapeutic points selected from the group consisting of LU7 (Lieque), LU10 (Yuji), LU11 (Shaoshang), LI1 (Shangyang), LI4 (Hegu), LI11 (Quchi), LI20 (Yingxiang), ST6 (Jiache), ST7 (Xiaguan), ST25 (Tianshu), ST34 (Liangqiu), ST36 (Zusanli), ST37 (Shangjuxu), ST39 (Xiajuxu), ST40 (Fenglong), ST44 (Neiting), SP4 (Gongsun), SP6 (Sanyinjiao), SP8 (Diji), SP9 (Yinlingquan), SP10 (Xuehai), SP21 (Dabao), HT5 (Tongli), HT6 (Yinxi), HT9 (Shaochong), SI1 (Shaoze), S13 (Houxi), SI19 (Tinggong), BL2 (Cuanzhu), BL7 (Tongtian), BL10 (Tianzhu), BL13 (Feishu), BL15 (Xinshu), BL17 (Geshu), BL21 (Weishu), BL23 (Shenshu), BL60 (Kunlun), KI1 (Yongquan), K13 (Taixi), K15 (Shuiquan), K16 (Zhaohai), K17 (Fuliu), PC4 (Ximen), PC6 (Neiguan), PC7 (Daling), PC8 (Laogong), PC9 (Zhongchong), TE1 (Guanchong), TE3 (Zhongzhu), TE5 (Waiguan), TE6 (Zhigou), TE17 (Yifeng), TE21 (Ermen), TE23 (Sizhukong), GB2 (Tinghui), GB20 (Fengchi), GB24 (Riyue), GB25 (Jingmen), GB31 (Fengshi), GB34 (Yanglingquan), GB39 (Xuanzhong), GB41 (Zulinqi), LR3 (Taichong), LR13 (Zhangmen), GV1 (Changqiang), GV8 (Jinsuo), GV9 (Zhiyang), GV12 (Shenzhu), GV14 (Dazhui), GV15 (Yamen), GV20 (Baihui), GV23 (Shangxing), GV26 (Shuigou), CV3 (Zhongji), CV4 (Guanyuan), CV6 (Qihai), CV8 (Shenque), CV12 (Zhongwan), CV17 (Danzhong), CV22 (Tiantu), CV23 (Lianquan), CV24 (Chengjiang), EX-HN3 (Yintang), EX-HN5 (Taiyang), EX-B1 (Dingchuan), EX-UE11 (Shixuan) and EX-LE6 (Dannang).

In some embodiments, the disease in the method of treating a disease of a mammal subject is selected from the group consisting of fever, convulsions, diarrhea, constipation, coma, rectal prolapse, prostration, enuresis, cough, anuresis, asthma, renal colic, excessive phlegm, dysmenorrhea, hyperhidrosis, chest tightness, chest pain, night sweats, hypochondriac pain, dizziness, stiff neck, insomnia, excessive dreaming, pruritus, palpitations, eye pain, swollen eyes, cardialgia, nasal congestion, rhinorrhea, stomach pain, tinnitus, hearing loss, nausea, vomiting, halitosis, hiccups, toothache, jaundice, trismus, biliary colic, sore throat, abdominal distension, abdominal pain and aphasia.

In another aspect, the present invention provides a composition which stimulates a set of therapeutic points or one or more therapeutic points of a mammal subject. The composition comprises one or more TRP channel agonists selected from the group consisting of 2-aminoethoxydiphenol borate, borneol, bisandrographolide A, camphor, 1,8-cineole, citral, citrate, eugenol and 6-gingerol.

In some embodiments, the composition of the present invention, comprises one or more TRP channel agonists selected from the group consisting of 2-aminoethoxydiphenol borate, borneol, bisandrographolide A, camphor, 1,8-cineole, citral, citrate, eugenol, 6-gingerol, and one or more penetration enhancing agents. In some embodiments, the penetration enhancing agent included in the composition of the present invention, is selected from the group consisting of terpinen-4-ol, α-terpineol and combinations thereof.

In some embodiments, the composition of the present invention, comprises one or more TRP channel agonists selected from the group consisting of 2-aminoethoxydiphenol borate, borneol, bisandrographolide A, camphor, 1,8-cineole, citral, citrate, eugenol, 6-gingerol, and one or two compounds selected from the group consisting of trans-caryophyllene and thujone.

In some embodiments, the composition of the present invention, comprises: one or more TRP channel agonists selected from the group consisting of 2-aminoethoxydiphenol borate, borneol, bisandrographolide A, camphor, 1,8-cineole, citral, citrate, eugenol and 6-gingerol; one or more penetration enhancing agents; and one or two compounds selected from the group consisting of trans-caryophyllene and thujone. In some embodiments, the penetration enhancing agent included in the composition of the present invention, is selected from the group consisting of terpinen-4-ol, α-terpineol and combinations thereof.

In some embodiments, the composition of the present invention, is applied topically as a patch. In some embodiments, the composition of the present invention, is applied topically in liquid form.

As used herein in the specification, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples are given by way of illustration only, since various changes and modifications within the spirit and scope of the invention will become apparent to those skilled in the art from this detailed description.

### DEFINITIONS

Throughout the present specification, the following terms have the indicated meaning:

The term “therapeutic action” as used herein refers to clinically desired results such as biological or chemical responses, alleviation or amelioration of one or more symptoms of a disease or condition, diminishment of extent of disease, stabilized state of disease, or improving the body functions.

The term “therapeutic point” as used herein refers to a specific site on the body of a mammal object, which can produce therapeutic action when appropriately stimulated. A therapeutic point in present invention can be an acupoint, “ah shi” point, trigger point and motor point.

The term “therapeutically effective amount” as used herein means that amount of an agent required to produce a clinically desired result such as a biological or chemical response, alleviation or amelioration of one or more symptoms of a disease or condition, diminishment of extent of disease, stabilized state of disease, or improving the body functions.

The term “pharmaceutically acceptable” used herein means that the substance or composition must be compatible chemically and/or toxicologically, with the other ingredients comprising a formulation and/or the mammal subject being treated therewith.

The terms “treating” and “treatment” as used herein mean the management and care of a patient for the purpose of combating a disease, disorder or condition, or improving the body functions of a mammal subject. The terms are intended to include the full spectrum of treatments for a given disorder from which the patient is suffering, such as the delaying of the progression of the disease, disorder or condition, the alleviation or relief of symptoms and complications, the prevention of the diseases, improving the body functions and/or the cure or elimination of the disease, disorder or condition. The patient to be treated is a mammal subject.

The term “optionally” used herein means that the subsequently described event(s) may or may not occur and includes both event(s) which occur and events that do not occur.

### DETAILED DESCRIPTION

Therapeutic points can be stimulated to produce the action potentials. The action potentials produced at therapeutic points can be transmitted by afferent nerve fibers to the central nervous system and cause the responses in a number of regions in the central nervous system, which may lead to the modulation of visceral functions and producing therapeutic action. Many methods are disclosed to stimulate therapeutic points to produce the action potentials, including needle insertion, applying an electric current, applying heat and applying other forms of energy. The present invention discloses a method to stimulate therapeutic points of a mammal subject to generate the action potentials, which are transmitted to the central nervous system and may produce therapeutic action. The method includes applying to a set of therapeutic points or one or more therapeutic points of a mammal subject a therapeutically effective amount of an agonist of a TRP channel selected from the group consisting of TRPV1, TRPV2, TRPV3, TRPV4, TRPA1, TRPM8, and combinations thereof.

Before the subject invention is further described, it is to be understood that the invention is not limited to the particular embodiments of the invention described below, as variations of the particular embodiments may be made and still fall within the scope of the appended claims. It is also to be understood that the terminology employed is for the purpose of describing particular embodiments and is not intended to be limiting. Instead, the scope of the present invention will be established by the appended claims.

Transient receptor potential (TRP) channels represent a superfamily of ion channels. In mammalian cells, TRPs are widely expressed in all tissue types. The nervous system contains most known TRP channel types. Mammalian TRP channels are encoded by six loosely related families of about 30 TRP genes. These families are grouped by homology and are named C, V, M, A, P and ML, representing canonical, vanilloid binding, melastatin related, ankyrin repeat, polycystin and mucolipin, respectively (see Clapham, D. E. “Transient receptor potential (TRP) channels.” In: Encyclopedia of Neuroscience (2009), vol. 9, pp. 1109-1133). TRP channels are composed of six-transmembrane (6TM) polypeptide subunits that assemble to form tetramers. These tetramers form pores in the membrane, allowing cations (e.g., Ca²⁺ and Na⁺) to permeate the membrane (see Clapham, D. E. TRP channels as cellular sensors. Nature, Vol. 426 (2003), pp. 517-524). TRP channel activation allows for entry of one or more cations into a cell and are involved in sensory transduction responding to a variety of stimuli.

Several TRP channels, including TRPA1, TRPM8, TRPV1, TRPV2, TRPV3 and TRPV4, are expressed in primary somatosensory neurons. They are activated at specific temperatures in a wide range. The activation of these TRP channels may lead to the generation of action potentials. The generated action potentials are transmitted to the central nervous system (see Vay, L. et al. The thermo-TRP ion channel family: properties and therapeutic implications. Br. J. Pharmacol., Vol. 165 (2012), pp. 787-801). These TRP channels are also activated by a variety of compounds, i.e., the agonists of TRP channels.

TRPA1 is a Ca²⁺-permeant, nonselective channel. It is activated by temperatures below 15° C. (see Clapham, D. E. TRP channels as cellular sensors. Nature, Vol. 426 (2003), pp. 517-524). It is activated by several compounds, such as allicin, eugenol, gingerol, linalool, etc. (Holzer, P. Transient receptor potential (TRP) channels as drug targets for diseases of the digestive system. Pharmacol. Ther., Vol. 131, no. 1 (2011), pp. 142-170).

TRPM8 is a Ca²⁺ permeable, nonselective channel. It can be activated by temperatures in the range roughly from 8 to 28° C. (Clapham, D. E. TRP channels as cellular sensors. Nature, Vol. 426 (2003), pp. 517-524). It is activated by several compounds, such as allicin, camphor, eugenol, 1,8-cineole, etc. (see Holzer, P. Transient receptor potential (TRP) channels as drug targets for diseases of the digestive system. Pharmacol. Ther., Vol. 131, no. 1 (2011), pp. 142-170; Selescu, T. et al. Camphor activates and sensitizes transient receptor potential melastatin 8 (TRPM8) to cooling and icilin. Chem. Senses., Vol. 38, no. 7 (2013), pp. 563-575; Vay, L. et al. The thermo-TRP ion channel family: properties and therapeutic implications. Br. J. Pharmacol., Vol. 165 (2012), pp. 787-801).

TRPV1 is a Ca²⁺-permeant channel. It is activated by temperatures above 43° C. It is activated by several compounds, such as allicin, camphor, eugenol, piperine, etc. (see Holzer, P. Transient receptor potential (TRP) channels as drug targets for diseases of the digestive system. Pharmacol. Ther., Vol. 131, no. 1 (2011), pp. 142-170; Clapham, D. E. TRP channels as cellular sensors. Nature, Vol. 426 (2003), pp. 517-524).

TRPV2 is a Ca²⁺-permeable cation channel. It is activated by temperatures above 53° C. It is activated by several compounds, such as 2-aminoethyoxydiphenyl borate, cannabidol, probenecid, etc. (see Holzer, P. Transient receptor potential (TRP) channels as drug targets for diseases of the digestive system. Pharmacol. Ther., Vol. 131, no. 1 (2011), pp. 142-170; Clapham, D. E. TRP channels as cellular sensors. Nature, Vol. 426 (2003), pp. 517-524).

TRPV3 is a Ca²⁺-permeable action channel. It is activated by elevated temperatures. It is activated by several compounds, such as borneol, camphor, carveol, 1,8-cineol, eugenol, thymol, etc. (see Holzer, P. Transient receptor potential (TRP) channels as drug targets for diseases of the digestive system. Pharmacol. Ther., Vol. 131, no. 1 (2011), pp. 142-170; Clapham, D. E. TRP channels as cellular sensors. Nature, Vol. 426 (2003), pp. 517-524).

TRPV4 is a Ca²⁺ and Mg²⁺ permeable cation channel. It is activated by elevated temperatures. It is activated by several compounds, such as citrate, 4α-phorbol 12,13-didecanoate, 5′,6′-epoxyeicosatrienoic acid, etc. (see Holzer, P. Transient receptor potential (TRP) channels as drug targets for diseases of the digestive system. Pharmacol. Ther., Vol. 131, no. 1 (2011), pp. 142-170; Clapham, D. E. TRP channels as cellular sensors. Nature, Vol. 426 (2003), pp. 517-524).

In one aspect, the present invention relates to a method of stimulating a set of therapeutic points or one or more therapeutic points of a mammal subject. The method includes applying to a set of therapeutic points or one or more therapeutic points of a mammal subject a therapeutically effective amount of an agonist of a TRP channel selected from the group consisting of TRPV1, TRPV2, TRPV3, TRPV4, TRPA1, TRPM8, and combinations thereof.

The TRP channel agonist applied in the method of stimulating a set of therapeutic points or one or more therapeutic points of a mammal subject, can be an agonist of one or more of TRPV1, TRPV2, TRPV3, TRPV4, TRPA1 and TRPM8. One or more TRP channel agonists can be applied. In some embodiments, two or more agonists that activate the same or different TRP channels are applied. In some embodiments, an agonist that activates two or more TRP channels is applied. The method described herein are not limited to any specific agonist. A variety of agonists of TRPV1, TRPV2, TRPV3, TRPV4, TRPA1 and TRPM8 can be used.

In some embodiments, the compositions employed in the method of stimulating a set of therapeutic points or one or more therapeutic points of a mammal subject, comprise one or more TRP channel agonists selected from the group consisting of 2-aminoethoxydiphenol borate, borneol, bisandrographolide A, camphor, 1,8-cineole, citral, citrate, eugenol and 6-gingerol. 2-Aminoethoxydiphenol borate is an agonist of TRPV1, TRPV2 and TRPV3 (see Holzer, P. Transient receptor potential (TRP) channels as drug targets for diseases of the digestive system. Pharmacol. Ther., Vol. 131, no. 1 (2011), pp. 142-170). Bisandrographolide A is an agonist of TRPV4 (see Smith, P. L. et al. Bisandrographolide from Andrographis paniculata activates TRPV4 channels. J. Biol. Chem., Vol. 281, no. 40 (2006), pp. 29897-29904). Borneol is an agonist of TRPV3 (see Holzer, P. Transient receptor potential (TRP) channels as drug targets for diseases of the digestive system. Pharmacol. Ther., Vol. 131, no. 1 (2011), pp. 142-170). Camphor is an agonist of TRPV1, TRPV3 and TRPM8 (Holzer, P. Transient receptor potential (TRP) channels as drug targets for diseases of the digestive system. Pharmacol. Ther., Vol. 131, no. 1 (2011), pp. 142-170; Selescu, T. et al. Camphor activates and sensitizes transient receptor potential melastatin 8 (TRPM8) to cooling and icilin. Chem. Senses., Vol. 38, no. 7 (2013), pp. 563-575). 1,8-Cineole is an agonist of TRPM8 (see Holzer, P. Transient receptor potential (TRP) channels as drug targets for diseases of the digestive system. Pharmacol. Ther., Vol. 131, no. 1 (2011), pp. 142-170). Citral is an agonist of TRPV1, TRPV3, TRPA1 and TRPM8 (see Holzer, P. Transient receptor potential (TRP) channels as drug targets for diseases of the digestive system. Pharmacol. Ther., Vol. 131, no. 1 (2011), pp. 142-170). Citrate is an agonist of TRPV4 (see Suzuki, M. et al. Impaired pressure sensation in mice lacking TRPV4. J. Biol. Chem., Vol. 278, no. 25 (2003), pp. 22664-22668). Eugenol is an agonist of TRPV1, TRPV3, TRPA1 and TRPM8 (see Holzer, P. Transient receptor potential (TRP) channels as drug targets for diseases of the digestive system. Pharmacol. Ther., Vol. 131, no. 1 (2011), pp. 142-170). 6-Gingerol is an agonist of TRPV1 and TRPA1 (see Dedov, V. N. et al. Gingerols: a novel class of vanilloid receptor (VR1) agonist. Br. J. Pharmacol., Vol. 137, no. 6 (2002), pp. 793-798; Bandell, M. et al. Noxious cold ion channel TRPA1 is activated by pungent compounds and bradykinin. Neuron, Vol. 41, no. 6 (2004), pp. 849-857).

The TRP channel agonist generally may be present in amounts of about 0.001% to 96% by weight of the compositions employed in the method of stimulating a set of therapeutic points or one or more therapeutic points of a mammal subject. In some embodiments, the TRP channel agonist is present in amounts of about 0.001% to 50% by weight of the compositions.

Besides 2-aminoethoxydiphenyl borate, camphor, citral, eugenol and 6-gingerol, exemplary agonists of TRPV1 that can be applied in accordance with the method of stimulating a set of therapeutic points or one or more therapeutic points of a mammal subject include, but are not limited to, allicin, allyl isothiocyanate, cannabidiol, capsaicin, evodiamine, geraniol, gingerols, isovelleral, 6-paradol, piperine, dehydropipernonaline, isochavicine, isopiperine, piperanine, pipermonaline, piperolein A, piperolein B, retrofractamide C, polygodial, shogaols, zingerone, brevetoxin, gambierol, resiniferatoxin, vanillotoxin DkTx, vanillotoxin VaTx1, vanillotoxin VaTx2, vanillotoxin VaTx3, acesulfame K, acids (pH<6), ammonia (pH>8), anandamide, clotrimazole, copper sulfate, ethanol, ferrous sulfate, 12-(S)-hydroperoxy eicosatetraenoic acid, 15-(S)-hydroperoxy eicosatetraenoic acid, leukotriene B4, N-arachidonoyl-dopamine, nitric oxide, nitro-oleic acid, N-oleoyldopamine, oleoylethanolamide, polyamines, putrescine, spermidine, spermine, propofol, saccharin, zinc sulfate, (6E)-N-[(4-hydroxy-3-methoxyphenyl)methyl]-8-methylnon-6-enamide, N-[(4-hydroxy-3-methoxy-phenyl)methyl]-8-methyl-(6E)-6-nonenamide, N-(3-methoxy-4-hydroxybenzyl)-8-methylnon tran-6-enamide, (E)-N-[(4-hydroxy-3-methoxyphenyl)methyl]-8-methyl-6-nonenamide, N-vanillyl-alkanedienamides, N-vanillyl-alkanedienyls, N-vanillyl-cis-monounsaturated alkenamides, dihydrocapsaicin, norhydrocapsaicin, nordihydrocapsaicin, homocapsaicin, homodihydrocapsaicin, warburganal, scutigeral, guaiacol, civamide, nonivamide, nuvanil, olvanil and tinyatoxin. Other TRPV1 agonists are vanilloids that have TRPV1 receptor-binding moieties such as mono-phenolic mono-substituted benzylamine amidated with an aliphatic cyclized, normal or branched substitution. See, e.g., Holzer, P. Transient receptor potential (TRP) channels as drug targets for diseases of the digestive system. Pharmacol. Ther., Vol. 131, no. 1 (2011), pp. 142-170, and U.S. Pat. No. 8,734,770. Additional exemplary TRPV1 agonists are described in U.S. Pat. Nos. 4,599,342, 5,962,532, 5,762,963, 5,221,692, 4,313,958, 4,532,139, 4,544,668, 4,564,633, 4,544,669, 4,493,848 and PCT publication WO 00/50387. The pharmaceutically acceptable salts of any of the above mentioned TRPV1 agonists may also be used. Any active geometric- or stereo-isomer of the forgoing agonists may be used.

Besides 2-aminoethoxydiphenyl borate, exemplary agonists of TRPV2 that can be applied in accordance with the method of stimulating a set of therapeutic points or one or more therapeutic points of a mammal subject include, but are not limited to, diphenylborinic anhydride (DPBA), Δ⁹-tetrahydrocannabinol (Δ⁹-THC or THC), cannabinol (CBN), cannabidiol (CBP), probenecid, O-1821, 11-hydroxy-Δ₉-tetrahydrocannabinol, nabilone, CP55940, HU-210, HU-211/dexanabinol, HU-331, HU-308, JWH-015, WINS 5,212-2, 2-Arachidonoylglycerol (2-AG), Arvil, PEA, AM404, O-1918 and JWH-133 (see U.S. Patent Application Publication No. 20130203715 and references therein). The pharmaceutically acceptable salts of any of the above mentioned TRPV2 agonists may also be used. Any active geometric- or stereo-isomer of the forgoing agonists may be used.

Besides 2-aminoethoxydiphenyl borate, borneol, camphor, 1,8-cineole, citral and eugenol, exemplary agonists of TRPV3 that can be applied in accordance with the method of stimulating a set of therapeutic points or one or more therapeutic points of a mammal subject include, but are not limited to, incensole, incensole acetate, a compound disclosed in U.S. Patent. Application Publication No. 2010/0099756 (e.g., a compound of Formula I or Formula II, compound IA), menthol, dihydrocarveol, carveol, thymol, vanillin, ethyl vanillin, cinnamaldehyde, diphenylamine (DPA), diphenylborinic anhydride (DPBA), (−)-isopinocampheol, (−)-fenchone, (−)-trans-pinocarveol, isoborneol, (+)-camphorquinone, 6-tert-butyl-m-cresol, carvacrol, p-xylenol, creosol, propofol, p-cymene, (−)-isopulegol, (−)-carvone, (+)-dihydrocarvone, (−)-menthone, (+)-linalool, geraniol, farnesyl pyrophosphate, farnesyl diphosphate, isopentenyl pyrophosphate and 1-isopropyl-4-methyl-bicyclo[3.1.0]hexan-4-ol (see U.S. Patent Application Publication No. 20130203715 and references therein). The pharmaceutically acceptable salts of any of the above mentioned TRPV3 agonists may also be used. Any active geometric- or stereo-isomer of the forgoing agonists may be used.

Besides bisandrographolide A and citrate, exemplary agonists of TRPV4 that can be applied in accordance with the method of stimulating a set of therapeutic points or one or more therapeutic points of a mammal subject include, but are not limited to, 4αPDD, GSK1016790A, 5′,6′-epoxyeicosatrienoic acid (5′6′-EET), 8′9′-epoxyeicosatrienoic (8′9′-EET), APP44-1, RN1747, arachidonic acid (AA), 12-O-tetradecanoylphorbol-13-acetate (TPA)/phorbol 12-myristate 13-acetate (PMA), compounds disclosed in WO 2006/029209 (e.g., a compound of Formula I, Ib, IHc, II, III, or IIIc, N-{(1S)-1-[({(4R)-1-[(4-chlorophenyl)sulfonyl]-3-oxohexahydro-1H-azepin-4-yl}amino)carbonyl]-3-methylbutyl}-1-benzothiophene-2-carboxamide, N-{(1S)-1-[({(4R)-1-[(4-fluorophenyl)sulfonyl]-3-oxohexahydro-1H-azepin-4-yl}amino)carbonyl]-3-methylbutyl}-1-benzothiophene-2-carboxamide, N-{(1S)-1-[({(4R)-1-[(2-cyanophenyl)sulfonyl]-3-oxohexahydro-1H-azepin-4-yl}amino)carbonyl]-3-methyl butyl}-1-methyl-1H-indole-2-carboxamide, N-{(1S)-1-[({(4R)-1-[(2-cyanophenyl)sulfonyl]hexahydro-1H-azepin-4-yl}amino)carbonyl]-3-methylbutyl}-1-methyl-1H-indole-2-carboxamide), N-(4-Hydroxyphenyl)-5Z,8Z,11Z,14Z-eicosatetraenamide (AM404) and anandamide (see U.S. Patent Application Publication No. 20130203715 and references therein). The pharmaceutically acceptable salts of any of the above mentioned TRPV4 agonists may also be used. Any active geometric- or stereo-isomer of the forgoing agonists may be used.

Besides citral, eugenol and 6-gingerol, exemplary agonists of TRPA1 that can be applied in accordance with the method of stimulating a set of therapeutic points or one or more therapeutic points of a mammal subject include, but are not limited to, allicin, allyl isothiocyanate (AITC), benzyl isothiocyanate (BITC), linalool, phenyl isothiocyanate, isopropyl isothiocyanate, methyl isothiocyanate, diallyl disulfide, acrolein (2-propenal), disulfiram (Antabuse®), farnesyl thiosalicylic acid (FTS), farnesyl thioacetic acid (FTA), chlordantoin (Sporostacin®, topical fungicidal), 15-d-PGJ2, 5, 8, 11, 14-eicosatetraynoic acid (ETYA), dibenzoazepine, dibenzoxazepine, dibenz-[b,f]-[1,4]-oxazepine (CR), 11H-dibenz[b,e]azepine, 1,2-naphthoquinone, 1,3-dihydroxynaphthalene, 2-methyl-1,4-naphthoquinone, 1-nitronaphthalene, hydroquinone, 4-phenyl-1,2-dihydronaphthalene, 3,5-ditert-butylphenol, 2,4-ditert-butylphenol, 1,3-butadiene, [(3E)-1-phenyl-1,3-pentadienyl]benzene, [(2Z)-3-phenyl-2-butenyl]benzene, mefenamic acid, flurbiprofen, ketoprofen, diclofenac, indomethacin, SC alkyne (SCA), pentenal, mustard oil alkyne (MOA), iodoacetamide, iodoacetamide alkyne, (2-aminoethyl)methanethiosulphonate (MTSEA), 4-hydroxy-2-noneal (HNE), 4-hydroxyhexenal (HHE), 2-chlorobenzalmalononitrile, N-chloro tosylamide (chloramine-T), aldehyde, acetaldehyde (U.S. Pat. No. 7,960,130), formaldehyde, o-anisaldehyde, isoflurane, isovelleral, hydrogen peroxide, URB597, thiosulfinate, flufenamic acid, niflumic acid, carvacrol, menthol, 6-shogoal, 6-paradol, propofol, thymol, 2-tert-butyl-5-methylphenol, icilin, methyl salicylate, arachidonic acid, cinnamaldehyde, super cinnamaldehyde, 10-hydroxy-2-decenoic acid, 10-hydroxydecanoic acid, 4-oxo-2-nonenal (4-ONE), 1-chloroacetophenone (CN), bromobenzyl cyanide, Compounds CA3, 13-19 and 21-27 from Defalco, J. et al. Oxime derivatives related to AP18: Agonists and antagonists of the TRPA1 receptor. Bioorg. Med. Chem. Lett., Vol. 20, no. 1 (2010), pp. 276-279, 2-chlorobenzylidene malononitrile (CS), morphanthridine, Compounds 6 and 32 from Gijsen, H. J. et al. Analogues of morphanthridine and the tear gas dibenz[b,f] [1,4]oxazepine (CR) as extremely potent activators of the human transient receptor potential ankyrin (TRPA1) channel. J. Med. Chem., Vol. 53, no. 19 (2010), pp. 7011-7020, methyl vinyl ketone, mesityl oxide, acrylic acid N-hydroxysuccinimide ester, hydrocinnamic acid N-hydroxysuccinimide ester, 3-(2-Pyridyldithio)propionic acid N-hydroxysuccinimide ester, N-acetyl-p-benzo-quinoneimine, 1T-acetoxychavicol acetate, piperine, isopiperine, isochavicine, piperanine, piperolein A, piperolein B, (2E,4E)-N-Isobutyl-2,4-decadienamide, nitro-oleic acid (OA-NO2), 2-chloroacetophenone, styrene, naphthalene, indolinone compounds (U.S. Patent Application Publication No. US 2011/0009379), tetrahydrocannabinol (THC or Δ⁹-THC), cannabidiol (CBD), cannabichromene (CBC), cannabinol (CBN), cannabigerol (CBG), THC acid (THC-A), tetrahydrocannabivarin (THCVA), CBD acid (CBD-A), Compound 1 (AMG5445), 4-methyl-N-[2,2,2-trichloro-1-(4-chlorophenylsulfanyl)ethyl]benzamide, N-[2,2,2-trichloro-1-(4-chlorophenylsulfanyl)ethyl]acetamid, AMG9090, AMG5445 and the compounds disclosed in U.S. Patent Application Publication No. 2010/0273773 (e.g., a compound of Formula I). See U.S. Patent Application Publication No. 20130203715 and references therein. The pharmaceutically acceptable salts of any of the above mentioned TRPA1 agonists may also be used. Any active geometric- or stereo-isomer of the forgoing agonists may be used.

Besides camphor, citral, 1,8-cineole and eugenol, exemplary agonists of TRPM8 that can be applied in accordance with the method of stimulating a set of therapeutic points or one or more therapeutic points of a mammal subject include, but are not limited to, L-carvone, geraniol, nerolidol, menthone, 1,4-cineole, terpineol, D-limonene, pulegol (e.g., isopulegol), carvacrol, linalool, menthol, icilin, trans-p-menthane-3,8-diol, cis-p-menthane-3,8-diol, N,2,3-trimethyl-2-isopropylbutanamide (WS-23), N-ethyl paramenthane-3-carboxamide (WS-3), menthone glycerin acetal (Frescolat MGA), menthoxypropanediol (Cooling agent 10), Coolact® P, PMD-38, monomenthyl succinate (Physcool), monomenthyl glutarate and hydroxycitronellal (see U.S. Pat. No. 7,727,516). The pharmaceutically acceptable salts of any of the above mentioned TRPM8 agonists may also be used. Any active geometric- or stereo-isomer of the forgoing agonists may be used.

In some embodiments, optionally in combination with any or all of above various embodiments, the compositions employed in the method of stimulating a set of therapeutic points or one or more therapeutic points of a mammal subject, further comprise one or more penetration enhancing agents. A penetration enhancing agent refers to an agent that enhances the penetration of an active agent into the skin and/or to the sites of action. In some embodiments, the penetration enhancing agent(s) included in the compositions that employed in the method of stimulating a set of therapeutic points or one or more therapeutic points of a mammal subject, is(are) terpinen-4-ol, or α-terpineol, or both.

Besides terpinen-4-ol and α-terpineol, other penetration enhancing agents suitable for use in the compositions employed in the method of stimulating a set of therapeutic points or one or more therapeutic points of a mammal subject include, but are not limited to, stearyl alcohol, oleyl alcohol, linoleyl alcohol, linolenyl alcohol, caprylic alcohol, decyl alcohol, lauryl alcohol, propylene glycol, polyethylene glycol, ethylene glycol, diethylene glycol, triethylene glycol, ethoxydiglycol, dipropylene glycol, glycerol, propanediol, butanediol, pentanediol, 1,2,6-hexanetriol, lauryl alcohol, myristyl alcohol, cetyl alcohol, capric acid, lauric acid, myristic acid, stearic acid, oleic acid, caprylic acid, valeric acid, heptanoic acid, pelargonic acid, caproic acid, isovaleric acid, neopentanoic acid, trimethyl hexanoic acid, neodecanoic acid, isostearic acid, neoheptanoic acid, neononanoic acid, isopropyl n-decanoate, isopropyl palmitate, octyldodecyl myristate, ethyl acetate, butyl acetate, methyl acetate, isopropyl n-butyrate, ethyl valerate, methyl propionate, diethyl sebacate, ethyl oleate, isopropyl n-hexanoate, isopropyl myristate, urea, dimethylacetamide, diethyltoluamide, dimethylformamide, dimethyloctamide, dimethyl decamide, 1-hexyl-4-methoxycarbonyl-2-pyrrolidone, 1-lauryl-4-carboxy-2-pyrrolidone, 1-methyl-4-carboxy-2-pyrrolidone, 1-alkyl-4-imidazolin-2-one, 1-methyl-2-pyrrolidone, 2-pyrrolidone, 1-lauryl-2-pyrrolidone, 1-hexyl-4-carboxy-2-pyrrolidone, 1-methyl-4-methoxycarbonyl-2-pyrrolidone, 1-lauryl-4-methoxycarbonyl-2-pyrrolidone, dimethyl sulfoxide, decyl methyl sulfoxide, N-cocoalkypyrrolidone, N-dimethylaminopropylpyrrolidone, N-tallowalkylpyrrolidone, N-cyclohexylpyrrolidone, 1-farnesylazacycloheptan-2-one, 1-geranylgeranylazacycloheptan-2-one, fatty acid esters of (2-hydroxyethyl)-2-pyrrolidone, 1-geranylazacycloheptan-2-one, 1-dodecylazacycloheptane-2-one (Azone®), 1-(3,7-dimethyloctylazacycloheptan-2-one, 1-geranylazacyclohexane-2-one, 1-(3,7,11-trimethyldodecyl)azacyclohaptan-2-one, 1-geranylazacyclopentan-2,5-dione, 1-farnesylazacyclopentan-2-one, benzyl alcohol, butanol, pentanol, hexanol, octanol, nonanol, decanol, ethanol, 2-butanol, 2-pentanol, propanol, diethanolamine, triethanolamine, hexamethylenelauramide and its derivatives, benzalkonium chloride, sodium laurate, sodium lauryl sulfate, cetylpyridinium chloride, citric acid, succinic acid, salicylic acid, salicylate, cetyl trimethyl ammonium bromide, tetradecyltrimethylammonium bromide, octadecyltrimethylammonium chloride, dodecyltrimethylammonium chloride, hexadecyltrimethylammonium chloride, Span 20, Span 40, Span 60, Span 80, Span 85, Poloxamer231, Poloxamer182, Poloxamer184), Brij 30, Brij 35, Brij 93, Brij 96, Span 99, Myrj45, Myrj51, Myrj52, Miglyol 840, glycholic, sodium salts of taurocholic, lecithin, desoxycholic acids, sodium cholate, D-limonene, α-pinene, β-carene, carvol, carvone, pulegone, piperitone, ylang-ylang, menthone, anise, chenopodium, limonene oxide, a-pinene oxide, cyclopentene oxide, cyclohexene oxide, N-heptane, N-octane, N-nonane, N-decane, N-undecane, N-dodecane, N-tridecane, N-tetradecane, N-hexadecane and essential oils (e.g., tea tree oils). See U.S. Pat. No. 8,734,770. Exemplary penetration enhancers are listed, for illustration and not limitation.

In some embodiments, optionally in combination with any or all of above various embodiments, the compositions employed in the method of stimulating a set of therapeutic points or one or more therapeutic points of a mammal subject, further comprise trans-caryophyllene, or thujone, or both. Trans-caryophyllene selectively binds to cannabinoid receptor type 2 (CB2). It is a functional CB2 agonist (see Gertsch, J. et al. Beta-caryophyllene is a dietary cannabinoid. Proc. Natl. Acad. Sci. USA, Vol. 105, no. 26 (2008), pp. 9099-9104). Trans-caryophyllene has neuroprotective activities and at least some its neuroprotective activities are due to the activation of CB2 receptor (see Chang, H. J. et al. Protective effect of β-caryophyllene, a natural bicyclic sesquiterpene, against cerebral ischemic injury. J. Med. Food, Vol. 16, no. 6 (2013), pp. 471-480; Choi, I. Y. et al. Activation of cannabinoid CB2 receptor-mediated AMPK/CREB pathway reduces cerebral ischemic injury. Am. J. Pathol., Vol. 182, no. 3 (2013), pp. 928-939). Thujone is a blocker of γ-aminobutyric acid type A (GABA_{A}) receptor (see Olsen, R. Absinthe and γ-aminobutyric acid receptors. Proc. Natl. Acad. Sci. USA, Vol. 97, no. 9 (2000), pp. 4417-4418). GABA_{A} receptors, when activated, selectively conduct Cl⁻ ion through their pores, resulting hyperpolarization of the neuron. This causes an inhibitory effect on neurotransmission by diminishing the chance of a successful action potential occurring. By inhibiting GABA_{A} receptors, neurons may be more easily fired. (see The Wormwood Society, The Shaky History of Thujone [online]).

In some embodiments, optionally in combination with any or all of above various embodiments, the compositions employed in the method of stimulating a set of therapeutic points or one or more therapeutic points of a mammal subject, are dissolved into a solvent. In some embodiments, the solvent is ethanol. In some embodiments, the solvent is water. In some embodiments, the solvent is ethanol water mixtures in which ethanol concentration ranges from 5% (w/w) to 95% (w/w). In some embodiments, the solvent is dimethyl sulfoxide. In some embodiments, the solvent comprises one or more components selected from the group consisting of glycerol tripalmitate, glycerol tristearate, triolein, trilinolein and trilinolenin. Glycerol tripalmitate and glycerol tristearate are not liquid at room temperature. Glycerol tripalmitate and glycerol tristearate are not used as the sole component of the solvent. However, a solvent that contains two or more components can include glycerol tripalmitate, or glycerol tristearate, or both, so long as glycerol tripalmitate, or glycerol tristearate, or both is (are) dissolved in the solvent.

In some embodiments, optionally in combination with any or all of above various embodiments, the compositions employed in the method of stimulating a set of therapeutic points or one or more therapeutic points of a mammal subject, are incorporated into carriers which may be a liquid, gel, ointment, cream, lotion, paste, polymer dispersions and polymer matrices. In some embodiments, the carrier is vegetable oil. In some embodiments, the carrier is soybean oil. In some embodiments, the carrier is tea seed oil. In some embodiments, the carrier is wheat germ oil. In some embodiments, the carrier is corn oil. In some embodiments, the carrier is rice bran oil. In some embodiments, the carrier is essential oil, i.e., ylang-ylang oil. Liquids that may be suitable for use as carriers in the compositions employed in the method of stimulating a set of therapeutic points or one or more therapeutic points of a mammal subject include, but are not limited to, apricot kernel oil, avocado oil, borage oil, canola oil, evening primrose oil, grapeseed oil, hazelnut oil, jojoba oil, rosa mosqueta oil, sweet almond oil and other essential oils (e.g., tea tree oil). In some embodiments, the carriers are water dispersions of one or more polymers that selected from the group consisting of polyacrylic acid, sodium polyacrylate, polyvinyl alcohol and sodium carboxymethyl cellulose. In some embodiments, the carriers are polymer matrices that are obtained from one or more polymers that selected from the group consisting of polyacrylic acid, sodium polyacrylate, polyvinyl alcohol and sodium carboxymethyl cellulose. Polymers whose dispersions and/or matrices are suitable for use as carriers in the compositions employed in the method of stimulating a set of therapeutic points or one or more therapeutic points of a mammal subject include, but are not limited to, ethylene vinyl acetate copolymers, silicon rubbers, polyurethanes, polyisobutylene (PIB), acrylic polymers, paraffin waxes, low-density polypropylene, styrene-butadiene copolymers, ethylene-ethacrylate copolymers, polyesters, polyamides, cellulose derivatives, chitosan, carrageenan and polyvinylpyrrolidone. See, for example, Valenta, C. et al. The use of polymers for dermal and transdermal delivery. Eur. J. Pharm., Biopharm., Vol. (58), no. 2 (2004), pp. 279-289.

In some embodiments, optionally in combination with any or all of above various embodiments, the compositions employed in the method of stimulating a set of therapeutic points or one or more therapeutic points of a mammal subject, are formulated as a liquid, patch, ointment, oil, cream, lotion, gel and paste.

In some embodiments, optionally in combination with any or all of above various embodiments, the compositions employed in the method of stimulating a set of therapeutic points or one or more therapeutic points of a mammal subject, are formulated as a patch. In some embodiments, the patches that contain the compositions employed in the method of stimulating a set of therapeutic points or one or more therapeutic points of a mammal subject, comprise a backing layer, a self-adhesive matrix and a protection layer to be removed before use of the patch. The backing layers can be therapeutic compound-permeable or therapeutic compound-impermeable. Suitable materials for a backing layer include, for example, a polyester film (e.g., 10˜20 μm thick), an ethylene-vinyl acetate copolymer, a polyamide, a woven fabric, a nonwoven fabric and the like. Suitable base polymers for a self-adhesive matrix are, for example, polyacrylates, polysiloxanes, polyurethanes or polyisobutylenes. Suitable materials for the protection layer include a polyester film, a piece of silicone paper and the like. Suitable films for the protection layer are supplied by a number of manufactures and known to the person skilled in the art. In some embodiments, the patches that contain the compositions employed in the method of stimulating a set of therapeutic points or one or more therapeutic points of a mammal subject, are produced by dispersing and dissolving the therapeutic compounds in a dispersion with stirring, adding the dispersion into a solution of polyacrylate or other polymers suitable for self-adhesive matrices and dispersing with stirring, coating the resulting dispersion onto a backing layer (the coating weight is 200-5000 g/m², preferably 250-2000 g/m²), laminating a removable protection layer onto the resulting product, cutting the patch into desired shape and size and packing to make finished products.

In some embodiments, optionally in combination with any or all of above various embodiments, the compositions employed in the method of stimulating a set of therapeutic points or one or more therapeutic points of a mammal subject, are applied to the mammal subject by topical route, subcutaneous route, transdermal route, transcutaneous route, intradermal route, intramuscular route and percutaneous route.

In some embodiments, optionally in combination with any or all of above various embodiments, the compositions employed in the method of stimulating a set of therapeutic points or one or more therapeutic points of a mammal subject, are applied to the mammal subject using one or more of iontophoresis, microdialysis, ultrafiltration, electromagnetics, osmosis, electroosmosis, sonophoresis, electroporation, thermal poration, microporation, skin permeabilization or laser.

In some embodiments, optionally in combination with any or all of above various embodiments, the compositions employed in the method of stimulating a set of therapeutic points or one or more therapeutic points of a mammal subject, are applied to the mammal subject using a device. The device configured to deliver the compositions can include one or more of a microneedle, a microfine lance, a microfine cannula, a microinjector, a jet fluid injector and a high pressure jet fluid injector. The device configured to deliver the compositions can include one or more of topical delivery devices, transdermal delivery devices, transcutaneous delivery devices, percutaneous delivery devices, intradermal delivery devices, intramuscular devices and subcutaneous devices.

In some embodiments, optionally in combination with any or all of above various embodiments, the compositions employed in the method of stimulating a set of therapeutic points or one or more therapeutic points of a mammal subject, are topically applied to the specified area in liquid form. In some embodiments, the compositions employed in the method of stimulating a set of therapeutic points or one or more therapeutic points of a mammal subject are topically applied to the specified area by drops or a spray mechanism.

In some embodiments, optionally in combination with any or all of above various embodiments, the compositions employed in the method of stimulating a set of therapeutic points or one or more therapeutic points of a mammal subject, are topically applied to the specified areas of the mammal subject by applying the patches that contain the compositions. In some embodiments, the compositions employed in the method of stimulating a set of therapeutic points or one or more therapeutic points of a mammal subject, are topically applied to the specified areas of the mammal subject by applying gels, or pastes, or creams, or ointments, or lotions that all contain the compositions.

The invention provides a method for stimulating a set of therapeutic points or one or more therapeutic points of a mammal subject that comprises applying a therapeutically effective amount of an agonist of TRPV1 channel to a set of therapeutic points or one or more therapeutic points of the mammal subject.

The invention provides a method for stimulating a set of therapeutic points or one or more therapeutic points of a mammal subject that comprises applying a therapeutically effective amount of an agonist of TRPV2 channel to a set of therapeutic points or one or more therapeutic points of the mammal subject.

The invention provides a method for stimulating a set of therapeutic points or one or more therapeutic points of a mammal subject that comprises applying a therapeutically effective amount of an agonist of TRPV3 channel to a set of therapeutic points or one or more therapeutic points of the mammal subject.

The invention provides a method for stimulating a set of therapeutic points or one or more therapeutic points of a mammal subject that comprises applying a therapeutically effective amount of an agonist of TRPV4 channel to a set of therapeutic points or one or more therapeutic points of the mammal subject.

The invention provides a method for stimulating a set of therapeutic points or one or more therapeutic points of a mammal subject that comprises applying a therapeutically effective amount of an agonist of TRPA1 channel to a set of therapeutic points or one or more therapeutic points of the mammal subject.

The invention provides a method for stimulating a set of therapeutic points or one or more therapeutic points of a mammal subject that comprises applying a therapeutically effective amount of an agonist of TRPM8 channel to a set of therapeutic points or one or more therapeutic points of the mammal subject.

In another aspect of the present invention, it is provided a method of treating a disease of a mammal subject. The method comprises: choosing a set of therapeutic points or one or more therapeutic points of the mammal subject based on subject's symptoms; and then applying to the chosen therapeutic point(s) of the subject a therapeutically effective amount of a composition comprising an agonist of a TRP channel selected from the group consisting of TRPV1, TRPV2, TRPV3, TRPV4, TRPA1, TRPM8, and combinations thereof.

The TRP channel agonist applied in the method of treating a disease of a mammal subject, can be an agonist of one or more of TRPV1, TRPV2, TRPV3, TRPV4, TRPA1 and TRPM8. One or more TRP channel agonists can be applied. In some embodiments, two or more agonists that activate the same or different TRP channels are applied. In some embodiments, an agonist that activates two or more TRP channels is applied. The method described herein are not limited to any specific agonist. A variety of agonists of TRPV1, TRPV2, TRPV3, TRPV4, TRPA1 and TRPM8 can be used.

Choosing therapeutic point(s) for disease treatment is well documented in several comprehensive books and is known to those skilled in the art (see for example, Shi, X. Acupuncture and moxibustion therapy (in Chinese). Beijing, People's Medical Publishing House, 2001).

In some embodiments, one or more therapeutic points selected from the group consisting of GV14 (Dazhui), LI11 (Quchi), LI4 (Hegu) and TE5 (Waiguan) are chosen to treat a fever.

In some embodiments, one or more therapeutic points selected from the group consisting of GV26 (Shuigou), CV24 (Chengjiang), LI4 (Hegu), LR3 (Taichong), GV8 (Jinsuo) and Yanglingquan (GB34) are chosen to treat convulsions.

In some embodiments, one or more therapeutic points selected from the group consisting of CV4 (Guanyuan), ST25 (Tianshu), ST36 (Zusanli), ST37 (Shangjuxu) and ST39 (Xiajuxu) are chosen to treat diarrhea.

In some embodiments, one or more therapeutic points selected from the group consisting of PC6 (Neiguan), TE6 (Zhigou), ST25 (Tianshu) and ST36 (Zusanli) are chosen to treat constipation.

In some embodiments, one or more acupoints selected from the group consisting of GV26 (Shuigou), EX-UE11 (Shixuan), LU11 (Shaoshang), LI1 (Shangyang), PC9 (Zhongchong), TE1 (Guanchong), HT9 (Shaochong), SI1 (Shaoze) and KI1 (Yongquan) are chosen for coma treatment.

In some embodiments, one or more therapeutic points selected from the group consisting of CV6 (Qihai), GV20 (Baihui), GV1 (Changqiang) and ST36 (Zusanli) are chosen to treat rectal prolapse.

In some embodiments, one or more therapeutic points selected from the group consisting of CV6 (Qihai), CV4 (Guanyuan), CV8 (Shenque), GV20 (Baihui), PC6 (Neiguan) and ST36 (Zusanli) are chosen for prostration treatments.

In some embodiments, one or more therapeutic points selected from the group consisting of CV4 (Guanyuan), SP6 (Sanyinjiao), BL23 (Shenshu) and ST36 (Zusanli) are chosen for enuresis treatment.

In some embodiments, one or more therapeutic points selected from the group consisting of LU7 (Lieque), GV12 (Shenzhu) and BL13 (Feishu) are chosen to treat a cough.

In some embodiments, one or more therapeutic points selected from the group consisting of CV3 (Zhongji), SP6 (Sanyinjiao), LI4 (Hegu) and SP9 (Yinlingquan) are chosen for anuresis treatment.

In some embodiments, one or more therapeutic points selected from the group consisting of CV22 (Tiantu), CV17 (Danzhong), BL13 (Feishu) and EX-B1 (Dingchuan) are chosen to treat asthma.

In some embodiments, one or more therapeutic points selected from the group consisting of GB25 (Jingmen), K15 (Shuiquan), BL23 (Shenshu) and SP6 (Sanyinjiao) are chosen to treat renal colic.

In some embodiments, one or more therapeutic points selected from the group consisting of CV12 (Zhongwan), ST40 (Fenglong) and ST36 (Zusanli) are chosen for the treatment of excessive phlegm.

In some embodiments, one or more therapeutic points selected from the group consisting of CV4 (Guanyuan), SP8 (Diji), SP6 (Sanyinjiao) and ST36 (Zusanli) are chosen to treat dysmenorrhea.

In some embodiments, one or more therapeutic points selected from the group consisting of L14 (Hegu) and K17 (Fuliu) are chosen to treat hyperhidrosis.

In some embodiments, one or more therapeutic points selected from the group consisting of PC6 (Neiguan), CV17 (Danzhong) and PC4 (Ximen) are chosen to treat chest tightness and chest pain.

In some embodiments, one or more therapeutic points selected from the group consisting of HT6 (Yinxi), S13 (Houxi) and K16 (Zhaohai) are chosen for night sweats treatments.

In some embodiments, one or more therapeutic points selected from the group consisting of TE6 (Zhigou), GB34 (Yanglingquan), SP21 (Dabao) and LR13 (Zhangmen) are chosen for hypochondriac pain treatments.

In some embodiments, one or more therapeutic points selected from the group consisting of GV20 (Baihui), EX-HN5 (Taiyang), LR3 (Taichong) and GB20 (Fengchi) are chosen to treat dizziness.

In some embodiments, one or more therapeutic points selected from the group consisting of GV14 (Dazhui), BL10 (Tianzhu), S13 (Houxi) and BL60 (Kunlun) are chosen to treat a stiff neck.

In some embodiments, one or more therapeutic points selected from the group consisting of GV20 (Baihui), EX-HN5 (Taiyang), K13 (Taixi), BL15 (Xinshu), BL23 (Shenshu) and SP6 (Sanyinjiao) are chosen to treat insomnia or excessive dreaming.

In some embodiments, one or more therapeutic points selected from the group consisting of SP10 (Xuehai), LI11 (Quchi), L14 (Hegu), LR3 (Taichong), SP6 (Sanyinjiao) and GB31 (Fengshi) are chosen to treat pruritus.

In some embodiments, one or more therapeutic points selected from the group consisting of PC6 (Neiguan), HT6 (Yinxi) and PC4 (Ximen) are chosen to treat palpitations.

In some embodiments, one or more therapeutic points selected from the group consisting of EX-HN3 (Yintang), BL2 (Cuanzhu), TE23 (Sizhukong) and EX-HN5 (Taiyang) are chosen to treat eye pain and swollen eyes.

In some embodiments, one or more therapeutic points selected from the group consisting of PC6 (Neiguan), PC4 (Ximen), CV17 (Danzhong) and PC7 (Daling) are chosen for cardialgia treatments.

In some embodiments, one or more therapeutic points selected from the group consisting of L120 (Yingxiang), EX-HN3 (Yintang), GV23 (Shangxing) and BL7 (Tongtian) are chosen to treat nasal congestion or rhinorrhea.

In some embodiments, one or more therapeutic points selected from the group consisting of CV12 (Zhongwan), ST34 (Liangqiu), ST36 (Zusanli) and BL21 (Weishu) are chosen to treat stomach pain.

In some embodiments, one or more therapeutic points selected from the group consisting of TE3 (Zhongzhu), TE5 (Waiguan), GB41 (Zulinqi), GB39 (Xuanzhong), GB20 (Fengchi), S119 (Tinggong), GB2 (Tinghui), TE21 (Ermen), TE17 (Yifeng), ST6 (Jiache) and ST7 (Xiaguan) are chosen for tinnitus or hearing loss treatments.

In some embodiments, one or more therapeutic points selected from the group consisting of PC6 (Neiguan), CV12 (Zhongwan) and ST36 (Zusanli) are chosen to treat nausea and vomiting.

In some embodiments, one or more therapeutic points selected from the group consisting of PC7 (Daling), PC8 (Laogong) and LI4 (Hegu) are chosen to treat halitosis.

In some embodiments, one or more therapeutic points selected from the group consisting of PC6 (Neiguan), CV12 (Zhongwan), CV22 (Tiantu), CV17 (Danzhong), TE17 (Yifeng), ST36 (Zusanli) and BL17 (Geshu) are chosen to treat hiccups.

In some embodiments, one or more therapeutic points selected from the group consisting of ST6 (Jiache), ST7 (Xiaguan), LI4 (Hegu) and ST44 (Neiting) are chosen to treat a toothache.

In some embodiments, one or more therapeutic points selected from the group consisting of GV9 (Zhiyang), LR3 (Taichong), GB34 (Yanglingquan) and ST36 (Zusanli) are chosen to treat jaundice.

In some embodiments, one or more therapeutic points selected from the group consisting of ST6 (Jiache), ST7 (Xiaguan), LI4 (Hegu), GV26 (Shuigou) and CV24 (Chengjiang) are chosen to treat trismus.

In some embodiments, one or more therapeutic points selected from the group consisting of GB24 (Riyue), LR3 (Taichong), GB34 (Yanglingquan) and EX-LE6 (Dannang) are chosen for biliary colic treatments.

In some embodiments, one or more therapeutic points selected from the group consisting of LU11 (Shaoshang), PC6 (Neiguan) and LI4 (Hegu) are chosen to treat a sore throat.

In some embodiments, one or more therapeutic points selected from the group consisting of CV12 (Zhongwan), PC6 (Neiguan), SP4 (Gongsun), ST36 (Zusanli), ST37 (Shangjuxu) and ST39 (Xiajuxu) are chosen to treat abdominal distension and abdominal pain.

In some embodiments, one or more therapeutic points selected from the group consisting of CV23 (Lianquan), LI4 (Hegu), GV15 (Ya men), PC6 (Neiguan) and HT5 (Tongli) are chosen to treat aphasia.

The location and function of some therapeutic points are listed below (see for example, World Health Organization Regional Office for the Western Pacific, WHO Standard Acupuncture Point Locations in the Western Pacific Region. Manila, Philippines, WPRO, 2008; Chang, D. Compilation of Extraordinary Acupoints (in Chinese). Beijing, People's Medical Publishing House, 1987):

LU7 (Lieque): located on the radial aspect of the forearm, between the tendons of the abductor pollicis longus and the extensor pollicis brevis muscles, in the groove for the abductor pollicis longus tendon, 1.5 B-cun superior to the palmar wrist crease. Stimulating this acupoint can treat headache, cough, nasal obstruction, sore throat, wrist pain, facial paralysis, stiff neck, enuresis and urinary retention.

LU10 (Yuji): located on the palm, radial to the middle point of the first metacarpal bone, at the border between the red and white flesh. Stimulating this acupoint can treat fever, cough, hemoptysis, aphonia, sore and swollen throat, wrist pain, bronchitis, bronchial asthma, night sweats and mastitis.

LU11 (Shaoshang): located on the thumb, radial to the distal phalanx, 0.1 F-cun proximal-lateral to the radial corner of the thumb nail, at the intersection of the vertical line of the radial border and the horizontal line of the base of the thumb nail. Stimulating this acupoint can treat sore and swollen throat, fever, bronchitis, bronchial asthma, parotitis and coma.

LI1 (Shangyang): located on the index finger, radial to the distal phalanx, 0.1 F-cun proximal-lateral to the radial corner of the index fingernail, at the intersection of the vertical line of the radial border of the fingernail and the horizontal line of the base of the index fingernail. Stimulating this acupoint can treat fever, sore and swollen throat, toothache, hives and coma after a stroke.

LI4 (Hegu): located on the dorsum of the hand, radial to the midpoint of the second metacarpal bone. Stimulating this acupoint can treat cold, headache, facial nerve paralysis, fever, diaphoresis, anhidrosis, night sweats, uncontrollable jerking of the limbs, neurosis, hysteria, epilepsy, lumbar sprain and stiff neck.

LI11 (Quchi): located on the lateral aspect of the elbow, at the midpoint of the line connecting LU5 with the lateral epicondyle of the humerus. Stimulating this acupoint can treat fever, heat stroke, hypertension, hives, goiter, elbow weakness, abdominal pain, vomiting and diarrhea.

LI20 (Yingxiang): located on the face, in the nasolabial sulcus, at the same level as the midpoint of lateral border of the ala of the nose. Stimulating this acupoint can treat nasal obstruction, epistaxis, facial nerve paralysis, facial swelling and facial itching.

ST6 (Jiache): located on the face, one fingerbreadth (middle finger) anterosuperior to the angle of the mandible. Stimulating this acupoint can treat facioplegia, toothache, parotitis, masticatory spasm and trigeminal neuralgia.

ST7 (Xiaguan): located on the face, in the depression between the midpoint of the inferior border of the zygomatic arch and the mandibular notch. Stimulating this acupoint can treat toothache, jaw arthritis, trigeminal neuralgia, hearing loss, tinnitus and sciatica.

ST25 (Tianshu): located on the upper abdomen, 2 B-cun lateral to the centre of the umbilicus. Stimulating this acupoint can treat acute and chronic enteritis, acute paralytic ileus, hysteria, cholecystitis, hepatitis, dysmenorrhea, endometritis and dysfunctional uterine bleeding.

ST34 (Liangqiu): located on the anterolateral aspect of the thigh, between the vastus lateralis muscle and the lateral border of the rectus femoris tendon, 2 B-cun superior to the base of the patella. Stimulating this acupoint can treat stomachache, mastitis and arthropathy.

ST36 (Zusanli): located on the anterior aspect of the leg, on the line connecting ST35 with ST41, 3 B-cun inferior to ST35. Stimulating this acupoint can treat stomachache, vomiting, diarrhea, malaria, liver diseases, gallbladder diseases, insomnia, hypertension, hyperlipidemia, coronary heart disease, angina pectoris, bronchitis, bronchial asthma, nephritis, cystitis, sexual impotence, spermatorrhea, irregular menstrual cycles, dysfunctional uterine bleeding, mastitis, hives, rheumatoid arthritis, shock and fever.

ST37 (Shangjuxu): located on the anterior aspect of the leg, on the line connecting ST35 with ST41, 6 B-cun inferior to ST35. Stimulating this acupoint can treat appendicitis, dysentery, chronic and acute enteritis, paralytic ileus, hepatitis, nephritis, arthritis and gastric ulcer.

ST39 (Xiajuxu): located on the anterior aspect of the leg, on the line connecting ST35 with ST41, 9 B-cun inferior to ST35. Stimulating this acupoint can treat chronic and acute enteritis, dysentery, hernia, paralysis, mastitis and intercostal neuralgia.

ST40 (Fenglong): located on the anterolateral aspect of the leg, lateral border of the tibialis anterior muscle, 8 B-cun superior to the prominence of the lateral malleolus. Stimulating this acupoint can treat asthma, dizziness, hepatitis, constipation, stiff neck, scapulohumeral periarthritis, arthritis, hypertension, hyperlipidemia, apoplexy sequela, obesity, nephritis, cystitis, urethritis, amenorrhea and dysfunctional uterine bleeding.

ST44 (Neiting): located on the dorsum of the foot, between the second and third toes, posterior to the web margin, at the border between the red and white flesh. Stimulating this acupoint can treat toothache, sore and swollen throat, nasal hemorrhage, abdominal distension, diarrhea and constipation.

SP4 (Gongsun): located on the medial aspect of the foot, anteroinferior to the base of the first metatarsal bone, at the border between the red and white flesh. Stimulating this acupoint can treat hepatitis, myocarditis, pleurisy, epilepsy, stomachache, diarrhea, dysentery, vomiting and abdominal pain.

SP6 (Sanyinjiao): located on the tibial aspect of the leg, posterior to the medial border of the tibia, 3 B-cun superior to the prominence of the medial malleolus. Stimulating this acupoint can treat hypertension, insomnia, abdominal distension, diarrhea, heart disease, hemiplegia, eczema, hives and diseases of urinary system and reproductive system.

SP8 (Diji): located on the tibial aspect of the leg, posterior to the medial border of the tibia, 3 B-cun inferior to SP9. Stimulating this acupoint can treat abdominal pain, diarrhea, edema, irregular menstrual cycles, spermatorrhea, dysuria and dysmenorrhea.

SP9 (Yinlingquan): located on the tibial aspect of the leg, in the depression between the inferior border of the medial condyle of the tibia and the medial border of the tibia. Stimulating this acupoint can treat urinary retention, urinary tract infection, diarrhea, dysentery, edema, jaundice, spermatorrhea, irregular menstrual cycles, leg paralysis and knee joint pain.

SP10 (Xuehai): located on the anteromedial aspect of the thigh, on the bulge of the vastus medialis muscle, 2 B-cun superior to the medial end of the base of the patella. Stimulating this acupoint can treat irregular menstrual cycles, dysmenorrhea, amenorrhea, dysfunctional uterine bleeding, hematuria, hives, eczema, pruritus, hypertension and malaria.

SP21 (Dabao): located in the lateral thoracic region, in the sixth intercostal space, on the midaxillary line. Stimulating this acupoint can treat chest and hypochondrium pain, cough, asthma, generalized pains and general fatigue.

HT5 (Tongli): located on the anteromedial aspect of the forearm, radial to the flexor carpi ulnaris tendon, 1 B-cun proximal to the palmar wrist crease. Stimulating this acupoint can treat aphasia, bradycardia, angina pectoris, jaw arthritis, elbow pain and arm pain.

HT6 (Yinxi): located on the anteromedial aspect of the forearm, radial to the flexor carpi ulnaris tendon, 0.5 B-cun proximal to the palmar wrist crease. Stimulating this acupoint can treat cardialgia, palpitation, aphasia and hematemesis.

HT9 (Shaochong): located on the little finger, radial to the distal phalanx, 0.1 F-cun proximal-lateral to the radial corner of the little fingernail, at the intersection of the vertical line of the radial border of the nail and horizontal line of the base of the little fingernail. Stimulating this acupoint can treat palpitation, cardialgia, coma, sore and swollen throat and hypochondriac pain.

SI1 (Shaoze): located on the little finger, ulnar to the distal phalanx, 0.1 F-cun proximal-medial to the ulnar corner of the little fingernail, at the intersection of the vertical line of ulnar border of the nail and horizontal line of the base of the little fingernail. Stimulating this acupoint can treat headache, fever, coma after stroke, mastitis, hearing loss, conjunctivitis and cataracts.

S13 (Houxi): located on the dorsum of the hand, in the depression proximal to the ulnar side of the fifth metacarpophalangeal joint, at the border between the red and white flesh. Stimulating this acupoint can treat hearing loss, tinnitus, malaria, vertex headache, facial spasms, stiff neck, hysteria, hives and acute lumbar sprain.

S119 (Tinggong): located on the face, in the depression between the anterior border of the centre of the tragus and the posterior border of the condylar process of the mandible. Stimulating this acupoint can treat hearing loss, tinnitus, otitis media, aphonia, toothache and eclampsia.

BL2 (Cuanzhu): located on the head, in the depression at the medial end of the eyebrow. Stimulating this acupoint can treat headache, dizziness, trigeminal neuralgia, facioplegia, conjunctivitis (pinkeye), myopia, dacryocystitis, rhinitis and lumbar sprain.

BL7 (Tongtian): located on the head, 4 B-cun superior to the anterior hairline, 1.5 B-cun lateral to the anterior median line. Stimulating this acupoint can treat vertex headache, dizziness, nasal obstruction, epistaxis, bronchitis, bronchial asthma, stroke sequelae, trigeminal neuralgia and facioplegia.

BL10 (Tianzhu): located in the posterior region of the neck, at the same level as the superior border of the spinous process of the second cervical vertebra (C2), in the depression lateral to the trapezius muscle. Stimulating this acupoint can treat headache, dizziness, stiff neck, conjunctivitis, retinal hemorrhage, glaucoma, cervical spondylopathy, lumbar sprain, insomnia, amnesia, nasal obstruction, throat pain and throat swelling.

BL13 (Feishu): located in the upper back region, at the same level as the inferior border of the spinous process of the third thoracic vertebra (T3), 1.5 B-cun lateral to the posterior median line. Stimulating this acupoint can treat cough, asthma, pneumonia, sore and swollen throats, nasal obstruction, emphysema, whooping cough, nephritis and back pains.

BL15 (Xinshu): located in the upper back region, at the same level as the inferior border of the spinous process of the fifth thoracic vertebra (T5), 1.5 B-cun lateral to the posterior median line. Stimulating this acupoint can treat angina pectoris, arrhythmia, tachycardia, bradycardia, insomnia and forgetfulness.

BL17 (Geshu): located in the upper back region, at the same level as the inferior border of the spinous process of the seventh thoracic vertebra (T7), 1.5 B-cun lateral to the posterior median line. Stimulating this acupoint can treat chronic hemorrhagic diseases, acute biliary tract infection, hiccup, cough, asthma, hot flashes, diaphragmatic spasm, gastritis, hepatitis and headache.

BL21 (Weishu): located in the upper back region, at the same level as the inferior border of the spinous process of the 12th thoracic vertebra (T12), 1.5 B-cun lateral to the posterior median line. Stimulating this acupoint can treat gastritis, gastric ulcer, dysentery, hepatitis, diabetes, dyspepsia and chronic diarrhea.

BL23 (Shenshu): located in the lumbar region, at the same level as the inferior border of the spinous process of the second lumbar vertebra (L2), 1.5 B-cun lateral to the posterior median line. Stimulating this acupoint can treat spermatorrhea, sexual impotence, enuresis, urinary tract infection, urinary retention, irregular menstrual cycles, tinnitus, hearing loss, insomnia, dizziness, chronic diarrhea and chronic lumbar pain.

BL60 (Kunlun): located on the posterolateral aspect of the ankle, in the depression between the prominence of the lateral malleolus and the calcaneal tendon. Stimulating this acupoint can treat vertex headache, headache, dizziness, nasal hemorrhage and ankle disorders.

KI1 (Yongquan): located on the sole of the foot, in the deepest depression of the sole when the toes are flexed. Stimulating this acupoint can treat coma, shock, epilepsy, infantile convulsions, vertex headache, hypertension, dizziness, insomnia, palpitation, acute hives and stroke sequelae.

K13 (Taixi): located on the posteromedial aspect of the ankle, in the depression between the prominence of the medial malleolus and the calcaneal tendon. Stimulating this acupoint can treat dizziness, tinnitus, hypoxia, visual deterioration, toothache, sore and swollen throat, hemoptysis, neurasthenia, emphysema, chronic lumbago, chronic diarrhea, spermatorrhea, insomnia and ankle pain.

K15 (Shuiquan): located on the medial aspect of the foot, 1 B-cun inferior to K13, in the depression anterior to the calcaneal tuberosity. Stimulating this acupoint can treat irregular menstrual cycles, dysmenorrhea, urinary retention and abdominal pain.

K16 (Zhaohai): located on the medial aspect of the foot, 1 B-cun inferior to the prominence of the medial malleolus, in the depression inferior to the medial malleolus. Stimulating this acupoint can treat dry throat, aphasia, visual deterioration, irregular menstrual cycles, dysmenorrhea, urinary tract infection, epilepsy, rheumatoid arthritis and constipation.

K17 (Fuliu): located on the posteromedial aspect of the leg, anterior to the calcaneal tendon, 2 B-cun superior to the prominence of the medial malleolus. Stimulating this acupoint can treat urinary tract infection, edema, spermatorrhea, sexual impotence, night sweats and lumbago.

PC4 (Ximen): located on the anterior aspect of the forearm, between the tendons of the palmaris longus and the flexor carpi radialis, 5 B-cun proximal to the palmar wrist crease. Stimulating this acupoint can treat angina pectoris, tachycardia, bradycardia, arrhythmia, rheumatic heart disease, mastitis, pleurisy and hysteria.

PC6 (Neiguan): located on the anterior aspect of the forearm, between the tendons of the palmaris longus and the flexor carpi radialis, 2 B-cun proximal to the palmar wrist crease. Stimulating this acupoint can tachycardia, bradycardia, angina pectoris, arrhythmia, stomachache, vomiting, hiccup, apoplexy and hemiplegia.

PC7 (Daling): located on the anterior aspect of the wrist, between the tendons of palmaris longus and the flexor carpi radialis, on the palmar wrist crease. Stimulating this acupoint can treat tachycardia, angina pectoris, stomachache, vomiting, wrist joint pain and tonsillitis.

PC8 (Laogong): located on the palm of the hand, in the depression between the second and third metacarpal bones, proximal to the metacarpophalangeal joints. Stimulating this acupoint can treat heat stroke, precordial pain, schizophrenia and mouth ulcers.

PC9 (Zhongchong): located on the middle finger, at the centre of the tip of the middle finger. Stimulating this acupoint can treat coma, fever, heat stroke, angina pectoris, stiff tongue, cerebral hemorrhage and epilepsy.

TE1 (Guanchong): located on the ring finger, ulnar to the distal phalanx, 0.1 F-cun proximal to the ulnar corner of the fingernail, at the intersection of the vertical line of the ulnar side of the nail and the horizontal line of the base of the fingernail. Stimulating this acupoint can treat headache, fever, conjunctivitis, sore throat, speech impairments, tinnitus, hearing loss and cerebrovascular accident.

TE3 (Zhongzhu): located on the dorsum of the hand, between the fourth and fifth metacarpal bones, in the depression proximal to the fourth metacarpophalangeal joint. Stimulating this acupoint can treat hearing loss, tinnitus, sore and swollen throat, conjunctivitis, optic neuritis, headache, lumbago, scapulohumeral periarthritis and upper limb paralysis.

TE5 (Waiguan): located on the posterior aspect of the forearm, midpoint of the interosseous space between the radius and the ulna, 2 B-cun proximal to the dorsal wrist crease. Stimulating this acupoint can treat fever, parotitis, pneumonia, migraine, tinnitus, hearing loss, facial nerve paralysis, stiff neck, paralysis after stroke, lumbar sprain, radial nerve palsy, hypochondriac pain, elbow pain and arm pain.

TE6 (Zhigou): located on the posterior aspect of the forearm, midpoint of the interosseous space between the radius and the ulna, 3 B-cun proximal to the dorsal wrist crease. Stimulating this acupoint can treat fever, asthma, angina pectoris, lumbar sprain, jaundice and constipation.

TE17 (Yifeng): located in the anterior region of the neck, posterior to the ear lobe, in the depression anterior to the inferior end of the mastoid process. Stimulating this acupoint can treat tinnitus, hearing loss, toothache, hives and facial paralysis.

TE21 (Ermen): located on the face, in the depression between the supratragic notch and the condylar process of the mandible. Stimulating this acupoint can treat tinnitus, hearing loss, toothache and otitis media.

TE23 (Sizhukong): located on the head, in the depression at the lateral end of the eyebrow. Stimulating this acupoint can treat headache, facioplegia, toothache, strabismus, myopia, optic neuritis and optic nerve atrophy.

GB2 (Tinghui): located on the face, in the depression between the intertragic notch and the condylar process of the mandible. Stimulating this acupoint can treat hearing loss, tinnitus, otitis media, toothache, facioplegia and jaw arthritis.

GB20 (Fengchi): located in the anterior of the neck, inferior to the occipital bone, in the depression between the origins of sternocleidomastoid and the trapezius muscles. Stimulating this acupoint can treat colds, headache, insomnia, dizziness, hypertension, conjunctivitis (pinkeye), myopia, glaucoma, optic nerve atrophy, cerebral hemorrhage, stiff neck and heel pain.

GB24 (Riyue): located in the anterior thoracic region, in the seventh intercostal space, 4 B-cun lateral to the anterior median line. Stimulating this acupoint can treat hypochondriac pain, jaundice, vomiting, acid regurgitation, hiccup, gastritis, cholecystitis and hepatitis.

GB25 (Jingmen): located on the lateral abdomen, inferior to the free extremity of the 12th rib. Stimulating this acupoint can treat abdominal distension, lower abdominal pain, urinary retention, lumbar pain and diarrhea.

GB31 (Fengshi): located on the lateral aspect of the thigh, in the depression posterior to the iliotibial band where the tip of the middle finger rests, when standing up with the arms hanging alongside the thigh. Stimulating this acupoint can treat leg paralysis, hives, headache, tinnitus, hearing loss and hypochondriac pain.

GB34 (Yanglingquan): located on the fibular aspect of the leg, in the depression anterior and distal to the head of the fibula. Stimulating this acupoint can treat liver diseases, gallbladder diseases, hypochondriac pain, sciatica, hypertension, stiff neck and arthropathy.

GB39 (Xuanzhong): located on the fibular aspect of the leg, anterior to the fibula, 3 B-cun proximal to the prominence of the lateral malleolus. Stimulating this acupoint can treat stiff neck, hypochondriac pain, lumbar sprain, headache, tonsillitis, rhinitis, nasal hemorrhage and ankle disorders.

GB41 (Zulinqi): located on the dorsum of the foot, distal to the junction of the bases of the fourth and fifth metatarsal bones, in the depression lateral to the fifth extensor digitorum longus tendon. Stimulating this acupoint can treat migraine, dizziness, paralysis after stroke, dyspnea, irregular menstrual cycles, mastitis, hypochondriac pain and dorsum foot diseases.

LR3 (Taichong): located on the dorsum of the foot, between the first and second metatarsal bones, in the depression distal to the junction of the bases of the two bones, over the dorsalis pedis artery. Stimulating this acupoint can treat headache, dizziness, facial paralysis, epilepsy, fever seizures, infantile convulsions, hypochondriac pain, jaundice, trigeminal neuroglia, conjunctivitis, keratitis, glaucoma, pharyngitis, laryngitis, hepatitis, angina pectoris, gastritis, enteritis, cystitis, urethritis and mastitis.

LR13 (Zhangmen): located on the lateral abdomen, inferior to the free extremity of the 11th rib. Stimulating this acupoint can treat jaundice, hypochondriac pain, hepatosplenomegaly, indigestion, vomiting and diarrhea.

GV1 (Changqiang): located in the perineal region, inferior to the coccyx, midway between the tip of the coccyx and the anus. Stimulating this acupoint can treat spermatorrhea, sexual impotence, diarrhea, constipation, infantile convulsion and hematochezia.

GV8 (Jinsuo): located in the upper back region, in the depression inferior to the spinous process of the ninth thoracic vertebra (T9), on the posterior median line. Stimulating this acupoint can treat epilepsy, stomachache and jaundice.

GV9 (Zhiyang): located in the upper back region, in the depression inferior to the spinous process of the seventh thoracic vertebra (T7), on the posterior median line. Stimulating this acupoint can treat cough, asthma, spine pain and stiffness and lower back pain and stiffness.

GV12 (Shenzhu): located in the upper back region, in the depression inferior to the spinous process of the third thoracic vertebra (T3), on the posterior median line. Stimulating this acupoint can treat cough, asthma, whooping cough, mania and schizophrenia.

GV14 (Dazhui): located in the posterior region of the neck, in the depression inferior to the spinous process of the seventh cervical vertebra (C7), on the posterior median line. Stimulating this acupoint can treat fever, malaria, heat stroke, cough, asthma, emphysema, hepatitis, homeopathy, eczema, paralysis, shoulder and back pains, epilepsy and physical weakness.

GV15 (Yamen): located in the posterior region of the neck, in the depression superior to the spinous process of the second cervical vertebra (C2), on the posterior median line. Stimulating this acupoint can treat stiff tongue, voice loss, epilepsy, heaviness in head, cerebrovascular accident, hysteria, schizophrenia, cerebellar hypoplasia and spondylosis.

GV20 (Baihui): located on the head, 5 B-cun superior to the anterior hairline, on the anterior median line. Stimulating this acupoint can headache, dizziness, palpitation, forgetfulness, aphasia due to stroke, hysteria, syncope, mania, rectal prolapse and uterine prolapse.

GV23 (Shangxing): located on the head, 1 B-cun superior to the anterior hairline, on the anterior median line. Stimulating this acupoint can treat headache, dizziness, sore swollen eyelid, nasosinusitis, epilepsy and keratitis.

GV26 (Shuigou): located on the face, at the midpoint of the philtrum midline. Stimulating this acupoint can treat coma, syncope, trismus and pain in the lumbar vertebra.

CV3 (Zhongji): located on the lower abdomen, 4 B-cun inferior to the centre of the umbilicus, on the anterior median line. Stimulating this acupoint can treat dysuria, enuresis, irregular menstrual cycles, dysmenorrhea, edema, infertility, dysfunctional uterine bleeding, sexual impotence and premature ejaculation.

CV4 (Guanyuan): located on the lower abdomen, 3 B-cun inferior to the centre of the umbilicus, on the anterior median line. Stimulating this acupoint can irregular menstrual cycles, dysmenorrhea, spermatorrhea, sexual impotence, enuresis, dysentery, rectal prolapse, hematochezia, endometritis, physical weakness and dysfunctional uterine bleeding.

CV6 (Qihai): located on the lower abdomen, 1.5 B-cun inferior to the centre of the umbilicus, on the anterior median line. Stimulating this acupoint can treat abdominal distension and fullness, dysentery, diarrhea, irregular menstrual cycles, dysmenorrhea, spermatorrhea, physical weakness, bronchial asthma, gastritis and angina pectoris.

CV8 (Shenque): located on the upper abdomen, in the centre of the umbilicus. Stimulating this acupoint can treat borborygmus, edema, rectal prolapse and prostration.

CV12 (Zhongwan): located on the upper abdomen, 4 B-cun superior to the centre of the umbilicus, on the anterior median line. Stimulating this acupoint can treat stomachache, vomiting, hiccup, borborygmus, diarrhea, constipation, chronic hepatitis, cholecystitis, bronchial asthma, hysteria and schizophrenia.

CV17 (Danzhong): located in the anterior thoracic region, at the same levels as the fourth intercostal space, on the anterior median line. Stimulating this acupoint can treat asthma, cough, cardialgia, palpitations, mastitis and esophagitis.

CV22 (Tiantu): located in the anterior region of the neck, in the centre of the suprasternal fossa, on the anterior median line. Stimulating this acupoint can treat cough, asthma, throat pain, throat swelling, aphasia, laryngospasm, bronchiectasis and pneumonia.

CV23 (Lianquan): located in the anterior region of the neck, superior to superior border to thyroid cartilage, in the depression superior to the hyoid bone, on the anterior median line. Stimulating this acupoint can treat aphasia after stroke, mouth and tongue sores, pharyngitis, dysphagia, cough and asthma.

CV24 (Chengjiang): located on the face, in the depression in the centre of the mentolabial sulcus. Stimulating this acupoint can treat facial paralysis, facial swelling, toothache, mouth and tongue sores and infantile anorexia.

EX-HN3 (Yintang): located at the midpoint between the medial end of two eyebrows. Stimulating this acupoint can treat infantile convulsions, convulsions, long term headache, nasal diseases, ophthalmalgia and dizziness.

EX-HN5 (Taiyang): located at the temporal part of the head, in the depression 1 fingerbreadth posterior to the middle point between the lateral end of the eyebrow and the outer canthus. Stimulating this acupoint can treat headache, toothache, migraine, dizziness and sore swollen eyelid.

EX-B1 (Dingchuan): located in the posterior region of the neck, at the same level as the inferior border of the spinous process of the seventh cervical vertebra (C7), 0.5 B-cun lateral to the posterior median line. Stimulating this acupoint can treat stiff neck, cough and bronchial asthma.

EX-UE11 (Shixuan): located on the tips of ten fingers, 0.1 F-cun distal to the nails. Each fingertip has a point, totally ten points. Stimulating this acupoint can treat shock, coma, hyperpyrexia, heat shock, epilepsy, hysteria, infantile convulsions and numbness in fingers.

EX-LE6 (Dannang): located on the superior lateral aspect of the lower leg, 2 F-cun directly below the depression anterior and inferior to the head of the fibula. Stimulating this acupoint can treat acute and chronic cholecystitis, cholelithiasis, biliary colic, hypochondriac pain and leg paralysis.

B-cun (bone cun) is defined as follows: dividing the height of the human body into 75 equal units, then estimating the length and width of a certain part of the body according to such units. One such unit is equal to 1 B-cun. F-cun (Finger cun) is defined as follows: the distance between the ends of the two radial creases of the interphalangeal joints of the middle finger is taken as 1 F-cun (middle finger cun) when the thumb and the middle finger are flexed to form a circle. In addition, F-cun can also be estimated via thumb measurement or finger width measurement. One fingerbreadth (F-breadth) is equal to the width of the distal phalanx of the middle finger. This should be distinguished from the middle finger cun (see World Health Organization Regional Office for the Western Pacific. WHO Standard Acupuncture Point Locations in the Western Pacific Region. Manila, Philippines, WPRO, 2008).

In some embodiments, the compositions employed in the method of treating a disease of a mammal subject, comprise one or more TRP channel agonists selected from the group consisting of 2-aminoethoxydiphenol borate, borneol, bisandrographolide A, camphor, 1,8-cineole, citral, citrate, eugenol and 6-gingerol. 2-Aminoethoxydiphenol borate is an agonist of TRPV1, TRPV2 and TRPV3 (see Holzer, P. Transient receptor potential (TRP) channels as drug targets for diseases of the digestive system. Pharmacol. Ther., Vol. 131, no. 1 (2011), pp. 142-170). Bisandrographolide A is an agonist of TRPV4 (see Smith, P. L. et al. Bisandrographolide from Andrographis paniculata activates TRPV4 channels. J. Biol. Chem., Vol. 281, no. 40 (2006), pp. 29897-29904). Borneol is an agonist of TRPV3 (see Holzer, P. Transient receptor potential (TRP) channels as drug targets for diseases of the digestive system. Pharmacol. Ther., Vol. 131, no. 1 (2011), pp. 142-170). Camphor is an agonist of TRPV1, TRPV3 and TRPM8 (Holzer, P. Transient receptor potential (TRP) channels as drug targets for diseases of the digestive system. Pharmacol. Ther., Vol. 131, no. 1 (2011), pp. 142-170; Selescu, T. et al. Camphor activates and sensitizes transient receptor potential melastatin 8 (TRPM8) to cooling and icilin. Chem. Senses., Vol. 38, no. 7 (2013), pp. 563-575). 1,8-Cineole is an agonist of TRPM8 (see Holzer, P. Transient receptor potential (TRP) channels as drug targets for diseases of the digestive system. Pharmacol. Ther., Vol. 131, no. 1 (2011), pp. 142-170). Citral is an agonist of TRPV1, TRPV3, TRPA1 and TRPM8 (see Holzer, P. Transient receptor potential (TRP) channels as drug targets for diseases of the digestive system. Pharmacol. Ther., Vol. 131, no. 1 (2011), pp. 142-170). Citrate is an agonist of TRPV4 (see Suzuki, M. et al. Impaired pressure sensation in mice lacking TRPV4. J. Biol. Chem., Vol. 278, no. 25 (2003), pp. 22664-22668). Eugenol is an agonist of TRPV1, TRPV3, TRPA1 and TRPM8 (see Holzer, P. Transient receptor potential (TRP) channels as drug targets for diseases of the digestive system. Pharmacol. Ther., Vol. 131, no. 1 (2011), pp. 142-170). 6-Gingerol is an agonist of TRPV1 and TRPA1 (see Dedov, V. N. et al. Gingerols: a novel class of vanilloid receptor (VR1) agonist. Br. J. Pharmacol., Vol. 137, no. 6 (2002), pp. 793-798; Bandell, M. et al. Noxious cold ion channel TRPA1 is activated by pungent compounds and bradykinin. Neuron, Vol. 41, no. 6 (2004), pp. 849-857).

The TRP channel agonist generally may be present in amounts of about 0.001% to 96% by weight of the compositions employed in the method of treating a disease of a mammal subject. In some embodiments, the TRP channel agonist is present in amounts of about 0.001% to 50% by weight of the compositions.

Besides 2-aminoethoxydiphenyl borate, camphor, citral, eugenol and 6-gingerol, exemplary agonists of TRPV1 that can be applied in accordance with the method of treating a disease of a mammal subject include, but are not limited to, allicin, allyl isothiocyanate, cannabidiol, capsaicin, evodiamine, geraniol, gingerols, isovelleral, 6-paradol, piperine, dehydropipernonaline, isochavicine, isopiperine, piperanine, pipermonaline, piperolein A, piperolein B, retrofractamide C, polygodial, shogaols, zingerone, brevetoxin, gambierol, resiniferatoxin, vanillotoxin DkTx, vanillotoxin VaTx1, vanillotoxin VaTx2, vanillotoxin VaTx3, acesulfame K, acids (pH<6), ammonia (pH>8), anandamide, clotrimazole, copper sulfate, ethanol, ferrous sulfate, 12-(S)-hydroperoxy eicosatetraenoic acid, 15-(S)-hydroperoxy eicosatetraenoic acid, leukotriene B4, N-arachidonoyl-dopamine, nitric oxide, nitro-oleic acid, N-oleoyldopamine, oleoylethanolamide, polyamines, putrescine, spermidine, spermine, propofol, saccharin, zinc sulfate, (6E)-N-[(4-hydroxy-3-methoxyphenyl)methyl]-8-methylnon-6-enamide, N-[(4-hydroxy-3-methoxy-phenyl)methyl]-8-methyl-(6E)-6-nonenamide, N-(3-methoxy-4-hydroxybenzyl)-8-methylnon tran-6-enamide, (E)-N-[(4-hydroxy-3-methoxyphenyl)methyl]-8-methyl-6-nonenamide, N-vanillyl-alkanedienamides, N-vanillyl-alkanedienyls, N-vanillyl-cis-monounsaturated alkenamides, dihydrocapsaicin, norhydrocapsaicin, nordihydrocapsaicin, homocapsaicin, homodihydrocapsaicin, warburganal, scutigeral, guaiacol, civamide, nonivamide, nuvanil, olvanil and tinyatoxin. Other TRPV1 agonists are vanilloids that have TRPV1 receptor-binding moieties such as mono-phenolic mono-substituted benzylamine amidated with an aliphatic cyclized, normal or branched substitution. See, for example, Holzer, P. Transient receptor potential (TRP) channels as drug targets for diseases of the digestive system. Pharmacol. Ther., Vol. 131, no. 1 (2011), pp. 142-170, and U.S. Pat. No. 8,734,770. Additional exemplary TRPV1 agonists are described in U.S. Pat. Nos. 4,599,342, 5,962,532, 5,762,963, 5,221,692, 4,313,958, 4,532,139, 4,544,668, 4,564,633, 4,544,669, 4,493,848 and PCT publication WO 00/50387. The pharmaceutically acceptable salts of any of the above mentioned TRPV1 agonists may also be used. Any active geometric- or stereo-isomer of the forgoing agonists may be used.

Besides 2-aminoethoxydiphenyl borate, exemplary agonists of TRPV2 that can be applied in accordance with the method of treating a disease of a mammal subject include, but are not limited to, diphenylborinic anhydride (DPBA), Δ⁹-tetrahydrocannabinol (Δ⁹-THC or THC), cannabinol (CBN), cannabidiol (CBP), probenecid, O-1821, 11-hydroxy-Δ₉-tetrahydrocannabinol, nabilone, CP55940, HU-210, HU-211/dexanabinol, HU-331, HU-308, JWH-015, WINS 5,212-2, 2-Arachidonoylglycerol (2-AG), Arvil, PEA, AM404, 0-1918 and JWH-133 (see U.S. Patent Application Publication No. 20130203715 and references therein). The pharmaceutically acceptable salts of any of the above mentioned TRPV2 agonists may also be used. Any active geometric- or stereo-isomer of the forgoing agonists may be used.

Besides 2-aminoethoxydiphenyl borate, borneol, camphor, 1,8-cineole, citral and eugenol, exemplary agonists of TRPV3 that can be applied in accordance with the method of treating a disease of a mammal subject include, but are not limited to, incensole, incensole acetate, a compound disclosed in U.S. Patent. Application Publication No. 2010/0099756 (e.g., a compound of Formula I or Formula II, compound IA), menthol, dihydrocarveol, carveol, thymol, vanillin, ethyl vanillin, cinnamaldehyde, diphenylamine (DPA), diphenylborinic anhydride (DPBA), (−)-isopinocampheol, (−)-fenchone, (−)-trans-pinocarveol, isoborneol, (+)-camphorquinone, 6-tert-butyl-m-cresol, carvacrol, p-xylenol, creosol, propofol, p-cymene, (−)-isopulegol, (−)-carvone, (+)-dihydrocarvone, (−)-menthone, (+)-linalool, geraniol, farnesyl pyrophosphate, farnesyl diphosphate, isopentenyl pyrophosphate and 1-isopropyl-4-methyl-bicyclo[3.1.0]hexan-4-ol. See U.S. Patent Application Publication No. 20130203715 and references therein. The pharmaceutically acceptable salts of any of the above mentioned TRPV3 agonists may also be used. Any active geometric- or stereo-isomer of the forgoing agonists may be used.

Besides bisandrographolide A and citrate, exemplary agonists of TRPV4 that can be applied in accordance with the method of treating a disease of a mammal subject include, but are not limited to, 4αPDD, GSK1016790A, 5′,6′-epoxyeicosatrienoic acid (5′6′-EET), 8′9′-epoxyeicosatrienoic (8′9′-EET), APP44-1, RN1747, arachidonic acid (AA), 12-O-tetradecanoylphorbol-13-acetate (TPA)/phorbol 12-myristate 13-acetate (PMA) and compounds disclosed in WO 2006/029209 (e.g., a compound of Formula I, Ib, IHc, II, III, or IIIc, N-{(1S)-1[({(4R)-1-[(4-chlorophenyl)sulfonyl]-3-oxohexahydro-1H-azepin-4-yl}amino)carbonyl]-3-methylbutyl}-1-benzothiophene-2-carboxamide, N-{(1S)-1-[({(4R)-1-[(4-fluorophenyl)sulfonyl]-3-oxohexahydro-1H-azepin-4-yl}amino)carbonyl]-3-methylbutyl}-1-benzothiophene-2-carboxamide, N-{(1S)-1-[({(4R)-1-[(2-cyanophenyl)sulfonyl]-3-oxohexahydro-1H-azepin-4-yl}amino)carbonyl]-3-methyl butyl}-1-methyl-1H-indole-2-carboxamide, N-{(1S)-1-[({(4R)-1-[(2-cyanophenyl)sulfonyl]hexahydro-1H-azepin-4-yl}ami-no)carbonyl]-3-methylbutyl}-1-methyl-1H-indole-2-carboxamide), N-(4-Hydroxyphenyl)-5Z,8Z,11Z,14Z-eicosatetraenamide (AM404) and anandamide. See U.S. Patent Application Publication No. 20130203715 and references therein. The pharmaceutically acceptable salts of any of the above mentioned TRPV4 agonists may also be used. Any active geometric- or stereo-isomer of the forgoing agonists may be used.

Besides citral, eugenol and 6-gingerol, exemplary agonists of TRPA1 that can be applied in accordance with the method of treating a disease of a mammal subject include, but are not limited to, allicin, allyl isothiocyanate (AITC), benzyl isothiocyanate (BITC), linalool, phenyl isothiocyanate, isopropyl isothiocyanate, methyl isothiocyanate, diallyl disulfide, acrolein (2-propenal), disulfiram (Antabuse®), farnesyl thiosalicylic acid (FTS), farnesyl thioacetic acid (FTA), chlordantoin (Sporostacin®, topical fungicidal), 15-d-PGJ2, 5, 8, 11, 14-eicosatetraynoic acid (ETYA), dibenzoazepine, dibenzoxazepine, dibenz-[b,f]-[1,4]-oxazepine (CR), 11H-dibenz[b,e]azepine, 1,2-naphthoquinone, 1,3-dihydroxynaphthalene, 2-methyl-1,4-naphthoquinone, 1-nitronaphthalene, hydroquinone, 4-phenyl-1,2-dihydronaphthalene, 3,5-ditert-butylphenol, 2,4-ditert-butylphenol, 1,3-butadiene, [(3E)-1-phenyl-1,3-pentadienyl]benzene, [(2Z)-3-phenyl-2-butenyl]benzene, mefenamic acid, flurbiprofen, ketoprofen, diclofenac, indomethacin, SC alkyne (SCA), pentenal, mustard oil alkyne (MOA), iodoacetamide, iodoacetamide alkyne, (2-aminoethyl)methanethiosulphonate (MTSEA), 4-hydroxy-2-noneal (HNE), 4-hydroxyhexenal (HHE), 2-chlorobenzalmalononitrile, N-chloro tosylamide (chloramine-T), aldehyde, acetaldehyde (U.S. Pat. No. 7,960,130), formaldehyde, o-anisaldehyde, isoflurane, isovelleral, hydrogen peroxide, URB597, thiosulfinate, flufenamic acid, niflumic acid, carvacrol, menthol, 6-shogoal, 6-paradol, propofol, thymol, 2-tert-butyl-5-methylphenol, icilin, methyl salicylate, arachidonic acid, cinnamaldehyde, super cinnamaldehyde, 10-hydroxy-2-decenoic acid, 10-hydroxydecanoic acid, 4-oxo-2-nonenal (4-ONE), 1-chloroacetophenone (CN), bromobenzyl cyanide, Compounds CA3, 13-19 and 21-27 from Defalco, J. et al. Oxime derivatives related to AP18: Agonists and antagonists of the TRPA1 receptor. Bioorg. Med. Chem. Lett., Vol. 20, no. 1 (2010), pp. 276-279, 2-chlorobenzylidene malononitrile (CS), morphanthridine, Compounds 6 and 32 from Gijsen, H. J. et al. Analogues of morphanthridine and the tear gas dibenz[b,f] [1,4]oxazepine (CR) as extremely potent activators of the human transient receptor potential ankyrin (TRPA1) channel. J. Med. Chem., Vol. 53, no. 19 (2010), pp. 7011-7020, methyl vinyl ketone, mesityl oxide, acrylic acid N-hydroxysuccinimide ester, hydrocinnamic acid N-hydroxysuccinimide ester, 3-(2-Pyridyldithio)propionic acid N-hydroxysuccinimide ester, N-acetyl-p-benzo-quinoneimine, 1T-acetoxychavicol acetate, piperine, isopiperine, isochavicine, piperanine, piperolein A, piperolein B, (2E,4E)-N-Isobutyl-2,4-decadienamide, nitro-oleic acid (OA-NO2), 2-chloroacetophenone, styrene, naphthalene, indolinone compounds (U.S. Patent Application Publication No. US 2011/0009379), tetrahydrocannabinol (THC or Δ⁹-THC), cannabidiol (CBD), cannabichromene (CBC), cannabinol (CBN), cannabigerol (CBG), THC acid (THC-A), tetrahydrocannabivarin (THCVA), CBD acid (CBD-A), Compound 1 (AMG5445), 4-methyl-N-[2,2,2-trichloro-1-(4-chlorophenylsulfanyl)ethyl]benzamide, N-[2,2,2-trichloro-1-(4-chlorophenylsulfanyl)ethyl]acetamid, AMG9090, AMG5445 and the compounds disclosed in U.S. Patent Application Publication No. 2010/0273773 (e.g., a compound of Formula I). See U.S. Patent Application Publication No. 20130203715 and references therein. The pharmaceutically acceptable salts of any of the above mentioned TRPA1 agonists may also be used. Any active geometric- or stereo-isomer of the forgoing agonists may be used.

Besides camphor, citral, 1,8-cineole and eugenol, exemplary agonists of TRPM8 that can be applied in accordance with the method of treating a disease of a mammal subject include, but are not limited to, L-carvone, geraniol, nerolidol, menthone, 1,4-cineole, terpineol, D-limonene, pulegol (e.g., isopulegol), carvacrol, linalool, menthol, icilin, trans-p-menthane-3,8-diol, cis-p-menthane-3,8-diol, N,2,3-trimethyl-2-isopropylbutanamide (WS-23), N-ethyl paramenthane-3-carboxamide (WS-3), menthone glycerin acetal (Frescolat MGA), menthoxypropanediol (Cooling agent 10), Coolact® P, PMD-38, monomenthyl succinate (Physcool), monomenthyl glutarate and hydroxycitronellal (see U.S. Pat. No. 7,727,516). The pharmaceutically acceptable salts of any of the above mentioned TRPM8 agonists may also be used. Any active geometric- or stereo-isomer of the forgoing agonists may be used.

In some embodiments, the compositions employed in the method of treating a disease of a mammal subject, optionally in combination with any or all of above various embodiments, further comprise one or more penetration enhancing agents. A penetration enhancing agent refers to an agent that enhances the penetration of an active agent into the skin and/or to the sites of action. In some embodiments, the penetration enhancing agent(s) included in the compositions that employed in the method of treating a disease of a mammal subject, is (are) terpinen-4-ol, or α-terpineol, or both.

Besides terpinen-4-ol and α-terpineol, other penetration enhancing agents suitable for use in the compositions employed in the method of treating a disease of a mammal subject include, but are not limited to, stearyl alcohol, oleyl alcohol, linoleyl alcohol, linolenyl alcohol, caprylic alcohol, decyl alcohol, lauryl alcohol, propylene glycol, polyethylene glycol, ethylene glycol, diethylene glycol, triethylene glycol, ethoxydiglycol, dipropylene glycol, glycerol, propanediol, butanediol, pentanediol, 1,2,6-hexanetriol, lauryl alcohol, myristyl alcohol, cetyl alcohol, capric acid, lauric acid, myristic acid, stearic acid, oleic acid, caprylic acid, valeric acid, heptanoic acid, pelargonic acid, caproic acid, isovaleric acid, neopentanoic acid, trimethyl hexanoic acid, neodecanoic acid, isostearic acid, neoheptanoic acid, neononanoic acid, isopropyl n-decanoate, isopropyl palmitate, octyldodecyl myristate, ethyl acetate, butyl acetate, methyl acetate, isopropyl n-butyrate, ethyl valerate, methyl propionate, diethyl sebacate, ethyl oleate, isopropyl n-hexanoate, isopropyl myristate, urea, dimethylacetamide, diethyltoluamide, dimethylformamide, dimethyloctamide, dimethyl decamide, 1-hexyl-4-methoxycarbonyl-2-pyrrolidone, 1-lauryl-4-carboxy-2-pyrrolidone, 1-methyl-4-carboxy-2-pyrrolidone, 1-alkyl-4-imidazolin-2-one, 1-methyl-2-pyrrolidone, 2-pyrrolidone, 1-lauryl-2-pyrrolidone, 1-hexyl-4-carboxy-2-pyrrolidone, 1-methyl-4-methoxycarbonyl-2-pyrrolidone, 1-lauryl-4-methoxycarbonyl-2-pyrrolidone, dimethyl sulfoxide, decyl methyl sulfoxide, N-cocoalkypyrrolidone, N-dimethylaminopropylpyrrolidone, N-tallowalkylpyrrolidone, N-cyclohexylpyrrolidone, 1-farnesylazacycloheptan-2-one, 1-geranylgeranylazacycloheptan-2-one, fatty acid esters of (2-hydroxyethyl)-2-pyrrolidone, 1-geranylazacycloheptan-2-one, 1-dodecylazacycloheptane-2-one (Azone®), 1-(3,7-dimethyloctylazacycloheptan-2-one, 1-geranylazacyclohexane-2-one, 1-(3,7,11-trimethyldodecyl)azacyclohaptan-2-one, 1-geranylazacyclopentan-2,5-dione, 1-farnesylazacyclopentan-2-one, benzyl alcohol, butanol, pentanol, hexanol, octanol, nonanol, decanol, ethanol, 2-butanol, 2-pentanol, propanol, diethanolamine, triethanolamine, hexamethylenelauramide and its derivatives, benzalkonium chloride, sodium laurate, sodium lauryl sulfate, cetylpyridinium chloride, citric acid, succinic acid, salicylic acid, salicylate, cetyl trimethyl ammonium bromide, tetradecyltrimethylammonium bromide, octadecyltrimethylammonium chloride, dodecyltrimethylammonium chloride, hexadecyltrimethylammonium chloride, Span 20, Span 40, Span 60, Span 80, Span 85, Poloxamer231, Poloxamer182, Poloxamer184), Brij 30, Brij 35, Brij 93, Brij 96, Span 99, Myrj45, Myrj51, Myrj52, Miglyol 840, glycholic, sodium salts of taurocholic, lecithin, desoxycholic acids, sodium cholate, D-limonene, α-pinene, β-carene, carvol, carvone, pulegone, piperitone, ylang-ylang, menthone, anise, chenopodium, limonene oxide, α-pinene oxide, cyclopentene oxide, cyclohexene oxide, N-heptane, N-octane, N-nonane, N-decane, N-undecane, N-dodecane, N-tridecane, N-tetradecane, N-hexadecane and essential oils (e.g., tea tree oils) (see U.S. Pat. No. 8,734,770). Exemplary penetration enhancers are listed, for illustration and not limitation.

In some embodiments, optionally in combination with any or all of above various embodiments, the compositions employed in the method of treating a disease of a mammal subject, further comprise trans-caryophyllene, or thujone, or both. Trans-caryophyllene selectively binds to cannabinoid receptor type 2 (CB2). It is a functional CB2 agonist (see Gertsch, J. et al. Beta-caryophyllene is a dietary cannabinoid. Proc. Natl. Acad. Sci. USA, Vol. 105, no. 26 (2008), pp. 9099-9104). Trans-caryophyllene has neuroprotective activities and at least some its neuroprotective activities are due to the activation of CB2 receptor (see Chang, H. J. et al. Protective effect of β-caryophyllene, a natural bicyclic sesquiterpene, against cerebral ischemic injury. J. Med. Food, Vol. 16, no. 6 (2013), pp. 471-480; Choi, I. Y. et al. Activation of cannabinoid CB2 receptor-mediated AMPK/CREB pathway reduces cerebral ischemic injury. Am. J. Pathol., Vol. 182, no. 3 (2013), pp. 928-939). Thujone is a blocker of γ-aminobutyric acid type A (GABA_{A}) receptor (see Olsen, R. Absinthe and γ-aminobutyric acid receptors. Proc. Natl. Acad. Sci. USA, Vol. 97, no. 9 (2000), pp. 4417-4418). GABA_{A} receptors, when activated, selectively conduct Cl⁻ ion through their pores, resulting hyperpolarization of the neuron. This causes an inhibitory effect on neurotransmission by diminishing the chance of a successful action potential occurring. By inhibiting GABA_{A} receptors, neurons may be more easily fired (see The Wormwood Society, The Shaky History of Thujone [online]).

In some embodiments, optionally in combination with any or all of above various embodiments, the compositions employed in the method of treating a disease of a mammal subject, are dissolved into a solvent. In some embodiments, the solvent is ethanol. In some embodiments, the solvent is water. In some embodiments, the solvent is ethanol water mixtures in which ethanol concentration ranges from 5% (w/w) to 95% (w/w). In some embodiments, the solvent is dimethyl sulfoxide. In some embodiments, the solvent comprises one or more components selected from the group consisting of glycerol tripalmitate, glycerol tristearate, triolein, trilinolein and trilinolenin. Glycerol tripalmitate and glycerol tristearate are not liquid at room temperature. Glycerol tripalmitate and glycerol tristearate are not used as the sole component of the solvent. However, a solvent that contains two or more components can include glycerol tripalmitate, or glycerol tristearate, or both, so long as glycerol tripalmitate, or glycerol tristearate, or both is (are) dissolved in the solvent.

In some embodiments, optionally in combination with any or all of above various embodiments, the compositions employed in the method of treating a disease of a mammal subject, are incorporated into carriers which may be a liquid, gel, ointment, cream, lotion, paste, polymer dispersions and polymer matrices. In some embodiments, the carrier is vegetable oil. In some embodiments, the carrier is soybean oil. In some embodiments, the carrier is tea seed oil. In some embodiments, the carrier is wheat germ oil. In some embodiments, the carrier is corn oil. In some embodiments, the carrier is rice bran oil. In some embodiments, the carrier is essential oil, i.e., ylang-ylang oil. Liquids that may be suitable for use as carriers in the compositions employed in the method of treating a disease of a mammal subject include, but are not limited to, apricot kernel oil, avocado oil, borage oil, canola oil, evening primrose oil, grapeseed oil, hazelnut oil, jojoba oil, rosa mosqueta oil, sweet almond oil and other essential oils (e.g., tea tree oil). In some embodiments, the carriers are water dispersions of one or more polymers that selected from the group consisting of polyacrylic acid, sodium polyacrylate, polyvinyl alcohol and sodium carboxymethyl cellulose. In some embodiments, the carriers are polymer matrices that are obtained from one or more polymers that selected from the group consisting of polyacrylic acid, sodium polyacrylate, polyvinyl alcohol and sodium carboxymethyl cellulose. Polymers whose dispersions and/or matrices are suitable for use as carriers in the compositions employed in the method of treating a disease of a mammal subject include, but are not limited to, ethylene vinyl acetate copolymers, silicon rubbers, polyurethanes, polyisobutylene (PIB), acrylic polymers, paraffin waxes, low-density polypropylene, styrene-butadiene copolymers, ethylene-ethacrylate copolymers, polyesters, polyamides, cellulose derivatives, chitosan, carrageenan and polyvinylpyrrolidone. See, for example, Valenta, C. et al. The use of polymers for dermal and transdermal delivery. Eur. J. Pharm., Biopharm., Vol. (58), no. 2 (2004), pp. 279-289.

In some embodiments, optionally in combination with any or all of above various embodiments, the compositions employed in the method of treating a disease of a mammal subject, are formulated as a liquid, patch, ointment, oil, cream, lotion, gel and paste.

In some embodiments, optionally in combination with any or all of above various embodiments, the compositions employed in the method of treating a disease of a mammal subject, are formulated as a patch. In some embodiments, the patches that contain the compositions employed in the method of treating a disease of a mammal subject, comprise a backing layer, a self-adhesive matrix and a protection layer to be removed before use of the patch. The backing layers can be therapeutic compound-permeable or therapeutic compound-impermeable. Suitable materials for a backing layer include, for example, a polyester film (e.g., 10˜20 μm thick), an ethylene-vinyl acetate copolymer, a polyamide, a woven fabric, a nonwoven fabric and the like. Suitable base polymers for a self-adhesive matrix are, for example, polyacrylates, polysiloxanes, polyurethanes or polyisobutylenes. Suitable materials for the protection layer include a polyester film, a piece of silicone paper and the like. Suitable films for the protection layer are supplied by a number of manufactures and known to the person skilled in the art. In some embodiments, the patches that contain the compositions employed in the method of treating a disease of a mammal subject, are produced by dispersing and dissolving the therapeutic compounds in a dispersion with stirring, adding the dispersion into a solution of polyacrylate or other polymers suitable for self-adhesive matrices and dispersing with stirring, coating the resulting dispersion onto a backing layer (the coating weight is 200-5000 g/m², preferably 250-2000 g/m²), laminating a removable protection layer onto the resulting product, cutting the patch into desired shape and size and packing to make finished products.

In some embodiments, optionally in combination with any or all of above various embodiments, the compositions employed in the method of treating a disease of a mammal subject, are applied to the mammal subject by topical route, subcutaneous route, transdermal route, transcutaneous route, intradermal route, intramuscular route and percutaneous route.

In some embodiments, optionally in combination with any or all of above various embodiments, the compositions employed in the method of treating a disease of a mammal subject, are applied to the mammal subject using one or more of iontophoresis, microdialysis, ultrafiltration, electromagnetics, osmosis, electroosmosis, sonophoresis, electroporation, thermal poration, microporation, skin permeabilization or laser.

In some embodiments, optionally in combination with any or all of above various embodiments, the compositions employed in the method of treating a disease of a mammal subject, are applied to the mammal subject using a device. The device configured to deliver the compositions can include one or more of a microneedle, a microfine lance, a microfine cannula, a microinjector, a jet fluid injector and a high pressure jet fluid injector. The device configured to deliver the compositions can include one or more of topical delivery devices, transdermal delivery devices, transcutaneous delivery devices, percutaneous delivery devices, intradermal delivery devices, intramuscular devices and subcutaneous devices.

In some embodiments, optionally in combination with any or all of above various embodiments, the compositions employed in the method of treating a disease of a mammal subject, are topically applied to the specified area in liquid form. In some embodiments, the compositions employed in the method of treating a disease of a mammal subject are topically applied to the specified area by drops or a spray mechanism.

In some embodiments, optionally in combination with any or all of above various embodiments, the compositions employed in the method of treating a disease of a mammal subject, are topically applied to the specified areas of the mammal subject by applying the patches that contain the compositions. In some embodiments, the compositions employed in the method of treating a disease of a mammal subject, are topically applied to the specified areas of the mammal subject by applying the gels, or pastes, or creams, or ointments, or lotions that all contain the compositions.

The invention provides a method of treating a disease of a mammal subject, comprising: choosing a set of therapeutic points or one or more therapeutic points of the mammal subject based on subject's symptoms; and then applying to the chosen therapeutic point(s) of the subject a therapeutically effective amount of an agonist of TRPV1 channel.

The invention provides a method of treating a disease of a mammal subject, comprising: choosing a set of therapeutic points or one or more therapeutic points of the mammal subject based on subject's symptoms; and then applying to the chosen therapeutic point(s) of the subject a therapeutically effective amount of an agonist of TRPV2 channel.

The invention provides a method of treating a disease of a mammal subject, comprising: choosing a set of therapeutic points or one or more therapeutic points of the mammal subject based on subject's symptoms; and then applying to the chosen therapeutic point(s) of the subject a therapeutically effective amount of an agonist of TRPV3 channel.

The invention provides a method of treating a disease of a mammal subject, comprising: choosing a set of therapeutic points or one or more therapeutic points of the mammal subject based on subject's symptoms; and then applying to the chosen therapeutic point(s) of the subject a therapeutically effective amount of an agonist of TRPV4 channel.

The invention provides a method of treating a disease of a mammal subject, comprising: choosing a set of therapeutic points or one or more therapeutic points of the mammal subject based on subject's symptoms; and then applying to the chosen therapeutic point(s) of the subject a therapeutically effective amount of an agonist of TRPA1 channel.

The invention provides a method of treating a disease of a mammal subject, comprising: choosing a set of therapeutic points or one or more therapeutic points of the mammal subject based on subject's symptoms; and then applying to the chosen therapeutic point(s) of the subject a therapeutically effective amount of an agonist of TRPM8 channel.

In another aspect, the present invention provides a composition for stimulating a set of therapeutic points or one or more therapeutic points of a mammal subject, comprising one or more TRP channel agonists selected from the group consisting of 2-aminoethoxydiphenol borate, borneol, bisandrographolide A, camphor, 1,8-cineole, citral, citrate, eugenol and 6-gingerol. 2-Aminoethoxydiphenol borate is an agonist of TRPV1, TRPV2 and TRPV3 (see Holzer, P. Transient receptor potential (TRP) channels as drug targets for diseases of the digestive system. Pharmacol. Ther., Vol. 131, no. 1 (2011), pp. 142-170). Bisandrographolide A is an agonist of TRPV4 (see Smith, P. L. et al. Bisandrographolide from Andrographis paniculata activates TRPV4 channels. J. Biol. Chem., Vol. 281, no. 40 (2006), pp. 29897-29904). Borneol is an agonist of TRPV3 (see Holzer, P. Transient receptor potential (TRP) channels as drug targets for diseases of the digestive system. Pharmacol. Ther., Vol. 131, no. 1 (2011), pp. 142-170). Camphor is an agonist of TRPV1, TRPV3 and TRPM8 (Holzer, P. Transient receptor potential (TRP) channels as drug targets for diseases of the digestive system. Pharmacol. Ther., Vol. 131, no. 1 (2011), pp. 142-170; Selescu, T. et al. Camphor activates and sensitizes transient receptor potential melastatin 8 (TRPM8) to cooling and icilin. Chem. Senses., Vol. 38, no. 7 (2013), pp. 563-575). 1,8-Cineole is an agonist of TRPM8 (see Holzer, P. Transient receptor potential (TRP) channels as drug targets for diseases of the digestive system. Pharmacol. Ther., Vol. 131, no. 1 (2011), pp. 142-170). Citral is an agonist of TRPV1, TRPV3, TRPA1 and TRPM8 (see Holzer, P. Transient receptor potential (TRP) channels as drug targets for diseases of the digestive system. Pharmacol. Ther., Vol. 131, no. 1 (2011), pp. 142-170). Citrate is an agonist of TRPV4 (see Suzuki, M. et al. Impaired pressure sensation in mice lacking TRPV4. J. Biol. Chem., Vol. 278, no. 25 (2003), pp. 22664-22668). Eugenol is an agonist of TRPV1, TRPV3, TRPA1 and TRPM8 (see Holzer, P. Transient receptor potential (TRP) channels as drug targets for diseases of the digestive system. Pharmacol. Ther., Vol. 131, no. 1 (2011), pp. 142-170). 6-Gingerol is an agonist of TRPV1 and TRPA1 (see Dedov, V. N. et al. Gingerols: a novel class of vanilloid receptor (VR1) agonist. Br. J. Pharmacol., Vol. 137, no. 6 (2002), pp. 793-798; Bandell, M. et al. Noxious cold ion channel TRPA1 is activated by pungent compounds and bradykinin. Neuron, Vol. 41, no. 6 (2004), pp. 849-857).

The TRP channel agonist generally may be present in amounts of about 0.001% to 96% by weight of the composition of the present invention. In some embodiments, the TRP channel agonist is present in amounts of about 0.001% to 50% by weight of the composition of the present invention.

Besides 2-aminoethoxydiphenyl borate, camphor, citral, eugenol and 6-gingerol, exemplary agonists of TRPV1 that can be applied in accordance with the composition of the present invention include, but are not limited to, allicin, allyl isothiocyanate, cannabidiol, capsaicin, evodiamine, geraniol, gingerols, isovelleral, 6-paradol, piperine, dehydropipernonaline, isochavicine, isopiperine, piperanine, pipermonaline, piperolein A, piperolein B, retrofractamide C, polygodial, shogaols, zingerone, brevetoxin, gambierol, resiniferatoxin, vanillotoxin DkTx, vanillotoxin VaTx1, vanillotoxin VaTx2, vanillotoxin VaTx3, acesulfame K, acids (pH<6), ammonia (pH>8), anandamide, clotrimazole, copper sulfate, ethanol, ferrous sulfate, 12-(5)-hydroperoxy eicosatetraenoic acid, 15-(S)-hydroperoxy eicosatetraenoic acid, leukotriene B4, N-arachidonoyl-dopamine, nitric oxide, nitro-oleic acid, N-oleoyldopamine, oleoylethanolamide, polyamines, putrescine, spermidine, spermine, propofol, saccharin, zinc sulfate, (6E)-N-[(4-hydroxy-3-methoxyphenyl)methyl]-8-methylnon-6-enamide, N-[(4-hydroxy-3-methoxy-phenyl)methyl]-8-methyl-(6E)-6-nonenamide, N-(3-methoxy-4-hydroxybenzyl)-8-methylnon tran-6-enamide, (E)-N-[(4-hydroxy-3-methoxyphenyl)methyl]-8-methyl-6-nonenamide, N-vanillyl-alkanedienamides, N-vanillyl-alkanedienyls, N-vanillyl-cis-monounsaturated alkenamides, dihydrocapsaicin, norhydrocapsaicin, nordihydrocapsaicin, homocapsaicin, homodihydrocapsaicin, warburganal, scutigeral, guaiacol, civamide, nonivamide, nuvanil, olvanil and tinyatoxin. Other TRPV1 agonists are vanilloids that have TRPV1 receptor-binding moieties such as mono-phenolic mono-substituted benzylamine amidated with an aliphatic cyclized, normal or branched substitution. See, e.g., Holzer, P. Transient receptor potential (TRP) channels as drug targets for diseases of the digestive system. Pharmacol. Ther., Vol. 131, no. 1 (2011), pp. 142-170, and U.S. Pat. No. 8,734,770. Additional exemplary TRPV1 agonists are described in U.S. Pat. Nos. 4,599,342, 5,962,532, 5,762,963, 5,221,692, 4,313,958, 4,532,139, 4,544,668, 4,564,633, 4,544,669, 4,493,848 and PCT publication WO 00/50387. The pharmaceutically acceptable salts of any of the above mentioned TRPV1 agonists may also be used. Any active geometric- or stereo-isomer of the forgoing agonists may be used.

Besides 2-aminoethoxydiphenyl borate, exemplary agonists of TRPV2 that can be applied in accordance with the composition of the present invention include, but are not limited to, diphenylborinic anhydride (DPBA), Δ⁹-tetrahydrocannabinol (Δ⁹-THC or THC), cannabinol (CBN), cannabidiol (CBP), probenecid, O-1821, 11-hydroxy-Δ₉-tetrahydrocannabinol, nabilone, CP55940, HU-210, HU-211/dexanabinol, HU-331, HU-308, JWH-015, WINS 5,212-2, 2-Arachidonoylglycerol (2-AG), Arvil, PEA, AM404, 0-1918 and JWH-133. See U.S. Patent Application Publication No. 2013/0203715 and references therein. The pharmaceutically acceptable salts of any of the above mentioned TRPV2 agonists may also be used. Any active geometric- or stereo-isomer of the forgoing agonists may be used.

Besides 2-aminoethoxydiphenyl borate, borneol, camphor, 1,8-cineole, citral and eugenol, exemplary agonists of TRPV3 that can be applied in accordance with the composition of the present invention include, but are not limited to, incensole, incensole acetate, a compound disclosed in U.S. Patent. Application Publication No. 2010/0099756 (e.g., a compound of Formula I or Formula II, compound IA), menthol, dihydrocarveol, carveol, thymol, vanillin, ethyl vanillin, cinnamaldehyde, diphenylamine (DPA), diphenylborinic anhydride (DPBA), (−)-isopinocampheol, (−)-fenchone, (−)-trans-pinocarveol, isoborneol, (+)-camphorquinone, 6-tert-butyl-m-cresol, carvacrol, p-xylenol, creosol, propofol, p-cymene, (−)-isopulegol, (−)-carvone, (+)-dihydrocarvone, (−)-menthone, (+)-linalool, geraniol, farnesyl pyrophosphate, farnesyl diphosphate, isopentenyl pyrophosphate and 1-isopropyl-4-methyl-bicyclo[3.1.0]hexan-4-ol. See, for example, U.S. Patent Application Publication No. 2013/0203715 and references therein. The pharmaceutically acceptable salts of any of the above mentioned TRPV3 agonists may also be used. Any active geometric- or stereo-isomer of the forgoing agonists may be used.

Besides bisandrographolide A and citrate, exemplary agonists of TRPV4 that can be applied in accordance with the composition of the present invention include, but are not limited to, 4αPDD, GSK1016790A, 5′,6′-epoxyeicosatrienoic acid (5′6′-EET), 8′9′-epoxyeicosatrienoic (8′9′-EET), APP44-1, RN1747, arachidonic acid (AA), 12-O-tetradecanoylphorbol-13-acetate (TPA)/phorbol 12-myristate 13-acetate (PMA) and compounds disclosed in WO 2006/029209 (e.g., a compound of Formula I, Ib, IHc, II, III, or IIIc, N-{(1S)-1-[({(4R)-1-[(4-chlorophenyl)sulfonyl]-3-oxohexahydro-1H-azepin-4-yl}amino)carbonyl]-3-methylbutyl}-1-benzothiophen-2-carboxamide, N-{(1S)-1-[({(4R)-1-[(4-fluorophenyl)sulfonyl]-3-oxohexahydro-1H-azepin-4-yl}amino)carbonyl]-3-methylbutyl}-1-benzothiophen-2-carboxamide, N-{(1S)-1-[({(4R)-1-[(2-cyanophenyl)sulfonyl]-3-oxohexahydro-1H-azepin-4-yl}amino)carbonyl]-3-methyl butyl}-1-methyl-1H-indole-2-carboxamide, N-{(1S)-1-[({(4R)-1-[(2-cyanophenyl)sulfonyl]hexahydro-1H-azepin-4-yl}amino)carbonyl]-3-methylbutyl}-1-methyl-1H-indole-2-carboxamide), N-(4-Hydroxyphenyl)-5Z,8Z,11Z,14Z-eicosatetraenamide (AM404) and anandamide. See U.S. Patent Application Publication No. 2013/0203715 and references therein. The pharmaceutically acceptable salts of any of the above mentioned TRPV4 agonists may also be used. Any active geometric- or stereo-isomer of the forgoing agonists may be used.

Besides citral, eugenol and 6-gingerol, exemplary agonists of TRPA1 that can be applied in accordance with the composition of the present invention include, but are not limited to, allicin, allyl isothiocyanate (AITC), benzyl isothiocyanate (BITC), linalool, phenyl isothiocyanate, isopropyl isothiocyanate, methyl isothiocyanate, diallyl disulfide, acrolein (2-propenal), disulfiram (Antabuse®), farnesyl thiosalicylic acid (FTS), farnesyl thioacetic acid (FTA), chlordantoin (Sporostacin®, topical fungicidal), 15-d-PGJ2, 5, 8, 11, 14-eicosatetraynoic acid (ETYA), dibenzoazepine, dibenzoxazepine, dibenz-[b,f]-[1,4]-oxazepine (CR), 11H-dibenz[b,e]azepine, 1,2-naphthoquinone, 1,3-dihydroxynaphthalene, 2-methyl-1,4-naphthoquinone, 1-nitronaphthalene, hydroquinone, 4-phenyl-1,2-dihydronaphthalene, 3,5-ditert-butylphenol, 2,4-ditert-butylphenol, 1,3-butadiene, [(3E)-1-phenyl-1,3-pentadienyl]benzene, [(2Z)-3-phenyl-2-butenyl]benzene, mefenamic acid, flurbiprofen, ketoprofen, diclofenac, indomethacin, SC alkyne (SCA), pentenal, mustard oil alkyne (MOA), iodoacetamide, iodoacetamide alkyne, (2-aminoethyl)methanethiosulphonate (MTSEA), 4-hydroxy-2-noneal (HNE), 4-hydroxyhexenal (HHE), 2-chlorobenzalmalononitrile, N-chloro tosylamide (chloramine-T), aldehyde, acetaldehyde (U.S. Pat. No. 7,960,130), formaldehyde, o-anisaldehyde, isoflurane, isovelleral, hydrogen peroxide, URB597, thiosulfinate, flufenamic acid, niflumic acid, carvacrol, menthol, 6-shogoal, 6-paradol, propofol, thymol, 2-tert-butyl-5-methylphenol, icilin, methyl salicylate, arachidonic acid, cinnamaldehyde, super cinnamaldehyde, 10-hydroxy-2-decenoic acid, 10-hydroxydecanoic acid, 4-oxo-2-nonenal (4-ONE), 1-chloroacetophenone (CN), bromobenzyl cyanide, Compounds CA3, 13-19 and 21-27 from Defalco, J. et al. Oxime derivatives related to AP18: Agonists and antagonists of the TRPA1 receptor. Bioorg. Med. Chem. Lett., Vol. 20, no. 1 (2010), pp. 276-279, 2-chlorobenzylidene malononitrile (CS), morphanthridine, Compounds 6 and 32 from Gijsen, H. J. et al. Analogues of morphanthridine and the tear gas dibenz[b,f] [1,4]oxazepine (CR) as extremely potent activators of the human transient receptor potential ankyrin (TRPA1) channel. J. Med. Chem., Vol. 53, no. 19 (2010), pp. 7011-7020, methyl vinyl ketone, mesityl oxide, acrylic acid N-hydroxysuccinimide ester, hydrocinnamic acid N-hydroxysuccinimide ester, 3-(2-Pyridyldithio)propionic acid N-hydroxysuccinimide ester, N-acetyl-p-benzo-quinoneimine, 1T-acetoxychavicol acetate, piperine, isopiperine, isochavicine, piperanine, piperolein A, piperolein B, (2E,4E)-N-Isobutyl-2,4-decadienamide, nitro-oleic acid (OA-NO2), 2-chloroacetophenone, styrene, naphthalene, indolinone compounds (U.S. Patent Application Publication No. 2011/0009379), tetrahydrocannabinol (THC or Δ⁹-THC), cannabidiol (CBD), cannabichromene (CBC), cannabinol (CBN), cannabigerol (CBG), THC acid (THC-A), tetrahydrocannabivarin (THCVA), CBD acid (CBD-A), Compound 1 (AMG5445), 4-methyl-N-[2,2,2-trichloro-1-(4-chlorophenylsulfanyl)ethyl]benzamide, N-[2,2,2-trichloro-1-(4-chlorophenylsulfanyl)ethyl]acetamid, AMG9090, AMG5445 and the compounds disclosed in U.S. Patent Application Publication No. 2010/0273773 (e.g., a compound of Formula I) (see U.S. Patent Application Publication No. 2013/0203715 and references therein). The pharmaceutically acceptable salts of any of the above mentioned TRPA1 agonists may also be used. Any active geometric- or stereo-isomer of the forgoing agonists may be used.

Besides camphor, citral, 1,8-cineole and eugenol, exemplary agonists of TRPM8 that can be applied in accordance with the composition of the present invention include, but are not limited to, L-carvone, geraniol, nerolidol, menthone, 1,4-cineole, terpineol, D-limonene, pulegol (e.g., isopulegol), carvacrol, linalool, menthol, icilin, trans-p-menthane-3,8-diol, cis-p-menthane-3,8-diol, N,2,3-tri methyl-2-isopropylbutanamide (WS-23), N-ethyl paramenthane-3-carboxamide (WS-3), menthone glycerin acetal (Frescolat MGA), menthoxypropanediol (Cooling agent 10), Coolact® P, PMD-38, monomenthyl succinate (Physcool), monomenthyl glutarate and hydroxycitronellal (see U.S. Pat. No. 7,727,516). The pharmaceutically acceptable salts of any of the above mentioned TRPM8 agonists may also be used. Any active geometric- or stereo-isomer of the forgoing agonists may be used.

In some embodiments, the composition of the present invention, optionally in combination with any or all of above various embodiments, further comprises one or more penetration enhancing agents. A penetration enhancing agent refers to an agent that enhances the penetration of an active agent into the skin and/or to the sites of action. In some embodiments, the penetration enhancing agent(s) included in the composition of the present invention, is (are) terpinen-4-ol, or α-terpineol, or both.

Besides terpinen-4-ol and α-terpineol, other penetration enhancing agents suitable for use in the composition of present invention include, but are not limited to, stearyl alcohol, oleyl alcohol, linoleyl alcohol, linolenyl alcohol, caprylic alcohol, decyl alcohol, lauryl alcohol, propylene glycol, polyethylene glycol, ethylene glycol, diethylene glycol, triethylene glycol, ethoxydiglycol, dipropylene glycol, glycerol, propanediol, butanediol, pentanediol, 1,2,6-hexanetriol, lauryl alcohol, myristyl alcohol, cetyl alcohol, capric acid, lauric acid, myristic acid, stearic acid, oleic acid, caprylic acid, valeric acid, heptanoic acid, pelargonic acid, caproic acid, isovaleric acid, neopentanoic acid, trimethyl hexanoic acid, neodecanoic acid, isostearic acid, neoheptanoic acid, neononanoic acid, isopropyl n-decanoate, isopropyl palmitate, octyldodecyl myristate, ethyl acetate, butyl acetate, methyl acetate, isopropyl n-butyrate, ethyl valerate, methyl propionate, diethyl sebacate, ethyl oleate, isopropyl n-hexanoate, isopropyl myristate, urea, dimethylacetamide, diethyltoluamide, dimethylformamide, dimethyloctamide, dimethyl decamide, 1-hexyl-4-methoxycarbonyl-2-pyrrolidone, 1-lauryl-4-carboxy-2-pyrrolidone, 1-methyl-4-carboxy-2-pyrrolidone, 1-alkyl-4-imidazolin-2-one, 1-methyl-2-pyrrolidone, 2-pyrrolidone, 1-lauryl-2-pyrrolidone, 1-hexyl-4-carboxy-2-pyrrolidone, 1-methyl-4-methoxycarbonyl-2-pyrrolidone, 1-lauryl-4-methoxycarbonyl-2-pyrrolidone, dimethyl sulfoxide, decyl methyl sulfoxide, N-cocoalkypyrrolidone, N-dimethylaminopropylpyrrolidone, N-tallowalkylpyrrolidone, N-cyclohexylpyrrolidone, 1-farnesylazacycloheptan-2-one, 1-geranylgeranylazacycloheptan-2-one, fatty acid esters of (2-hydroxyethyl)-2-pyrrolidone, 1-geranylazacycloheptan-2-one, 1-dodecylazacycloheptane-2-one (Azone®), 1-(3,7-dimethyloctylazacycloheptan-2-one, 1-geranylazacyclohexane-2-one, 1-(3,7,11-trimethyldodecyl)azacyclohaptan-2-one, 1-geranylazacyclopentan-2,5-dione, 1-farnesylazacyclopentan-2-one, benzyl alcohol, butanol, pentanol, hexanol, octanol, nonanol, decanol, ethanol, 2-butanol, 2-pentanol, propanol, diethanolamine, triethanolamine, hexamethylenelauramide and its derivatives, benzalkonium chloride, sodium laurate, sodium lauryl sulfate, cetylpyridinium chloride, citric acid, succinic acid, salicylic acid, salicylate, cetyl trimethyl ammonium bromide, tetradecyltrimethylammonium bromide, octadecyltrimethylammonium chloride, dodecyltrimethylammonium chloride, hexadecyltrimethylammonium chloride, Span 20, Span 40, Span 60, Span 80, Span 85, Poloxamer231, Poloxamer182, Poloxamer184), Brij 30, Brij 35, Brij 93, Brij 96, Span 99, Myrj45, Myrj51, Myrj52, Miglyol 840, glycholic, sodium salts of taurocholic, lecithin, desoxycholic acids, sodium cholate, D-limonene, α-pinene, β-carene, carvol, carvone, pulegone, piperitone, ylang-ylang, menthone, anise, chenopodium, limonene oxide, α-pinene oxide, cyclopentene oxide, cyclohexene oxide, N-heptane, N-octane, N-nonane, N-decane, N-undecane, N-dodecane, N-tridecane, N-tetradecane, N-hexadecane and essential oils (e.g., tea tree oils). See U.S. Pat. No. 8,734,770. Exemplary penetration enhancers are listed, for illustration and not limitation.

In some embodiments, optionally in combination with any or all of above various embodiments, the composition of the present invention, further comprises trans-caryophyllene, or thujone, or both. Trans-caryophyllene selectively binds to cannabinoid receptor type 2 (CB2). It is a functional CB2 agonist (see Gertsch, J. et al. Beta-caryophyllene is a dietary cannabinoid. Proc. Natl. Acad. Sci. USA, Vol. 105, no. 26 (2008), pp. 9099-9104). Trans-caryophyllene has neuroprotective activities and at least some its neuroprotective activities are due to the activation of CB2 receptor (see Chang, H. J. et al. Protective effect of β-caryophyllene, a natural bicyclic sesquiterpene, against cerebral ischemic injury. J. Med. Food, Vol. 16, no. 6 (2013), pp. 471-480; Choi, I. Y. et al. Activation of cannabinoid CB2 receptor-mediated AMPK/CREB pathway reduces cerebral ischemic injury. Am. J. Pathol., Vol. 182, no. 3 (2013), pp. 928-939). Thujone is a blocker of γ-aminobutyric acid type A (GABA_{A}) receptor (see Olsen, R. Absinthe and γ-aminobutyric acid receptors. Proc. Natl. Acad. Sci. USA, Vol. 97, no. 9 (2000), pp. 4417-4418). GABA_{A} receptors, when activated, selectively conduct Cl⁻ ion through their pores, resulting hyperpolarization of the neuron. This causes an inhibitory effect on neurotransmission by diminishing the chance of a successful action potential occurring. By inhibiting GABA_{A} receptors, neurons may be more easily fired (see The Wormwood Society, The Shaky History of Thujone [online]).

In some embodiments, optionally in combination with any or all of above various embodiments, the composition of the present invention, is dissolved into a solvent. In some embodiments, the solvent is ethanol. In some embodiments, the solvent is water. In some embodiments, the solvent is ethanol water mixtures in which ethanol concentration ranges from 5% (w/w) to 95% (w/w). In some embodiments, the solvent is dimethyl sulfoxide. In some embodiments, the solvent comprises one or more components selected from the group consisting of glycerol tripalmitate, glycerol tristearate, triolein, trilinolein and trilinolenin. Glycerol tripalmitate and glycerol tristearate are not liquid at room temperature. Glycerol tripalmitate and glycerol tristearate are not used as the sole component of the solvent. However, a solvent that contains two or more components can include glycerol tripalmitate, or glycerol tristearate, or both, so long as glycerol tripalmitate, or glycerol tristearate, or both is (are) dissolved in the solvent.

In some embodiments, optionally in combination with any or all of above various embodiments, the composition of the present invention, is incorporated into carriers which may be a liquid, gel, ointment, cream, lotion, paste, polymer dispersions and polymer matrices. In some embodiments, the carrier is vegetable oil. In some embodiments, the carrier is soybean oil. In some embodiments, the carrier is tea seed oil. In some embodiments, the carrier is wheat germ oil. In some embodiments, the carrier is corn oil. In some embodiments, the carrier is rice bran oil. In some embodiments, the carrier is essential oil, i.e., ylang-ylang oil. Liquids that may be suitable for use as carriers in the composition of the present invention include, but are not limited to, apricot kernel oil, avocado oil, borage oil, canola oil, evening primrose oil, grapeseed oil, hazelnut oil, jojoba oil, rosa mosqueta oil, sweet almond oil and other essential oils (e.g., tea tree oil). In some embodiments, the carriers are water dispersions of one or more polymers that selected from the group consisting of polyacrylic acid, sodium polyacrylate, polyvinyl alcohol and sodium carboxymethyl cellulose. In some embodiments, the carriers are polymer matrices that are obtained from one or more polymers that selected from the group consisting of polyacrylic acid, sodium polyacrylate, polyvinyl alcohol and sodium carboxymethyl cellulose. Polymers whose dispersions and/or matrices are suitable for use as carriers in the composition of the present invention include, but are not limited to, ethylene vinyl acetate copolymers, silicon rubbers, polyurethanes, polyisobutylene (PIB), acrylic polymers, paraffin waxes, low-density polypropylene, styrene-butadiene copolymers, ethylene-ethacrylate copolymers, polyesters, polyamides, cellulose derivatives, chitosan, carrageenan and polyvinylpyrrolidone. See, for example, Valenta, C. et al. The use of polymers for dermal and transdermal delivery. Eur. J. Pharm., Biopharm., Vol. (58), no. 2 (2004), pp. 279-289.

In some embodiments, optionally in combination with any or all of above various embodiments, the composition of the present invention, is formulated as a liquid, patch, ointment, oil, cream, lotion, gel and paste.

In some embodiments, optionally in combination with any or all of above various embodiments, the composition of the present invention, is formulated as a patch. In some embodiments, the patches that contain the composition of the present invention, comprise a backing layer, a self-adhesive matrix and a protection layer to be removed before use of the patch. The backing layers can be therapeutic compound-permeable or therapeutic compound-impermeable. Suitable materials for a backing layer include, for example, a polyester film (e.g., 10˜20 μm thick), an ethylene-vinyl acetate copolymer, a polyamide, a woven fabric, a nonwoven fabric and the like. Suitable base polymers for a self-adhesive matrix are, for example, polyacrylates, polysiloxanes, polyurethanes or polyisobutylenes. Suitable materials for the protection layer include a polyester film, a piece of silicone paper and the like. Suitable films for the protection layer are supplied by a number of manufactures and known to the person skilled in the art. In some embodiments, the patches that contain the composition of the present invention, are produced by dispersing and dissolving the therapeutic compounds in a dispersion with stirring, adding the dispersion into a solution of polyacrylate or other polymers suitable for self-adhesive matrices and dispersing with stirring, coating the resulting dispersion onto a backing layer (the coating weight is 200-5000 g/m², preferably 250-2000 g/m²), laminating a removable protection layer onto the resulting product, cutting the patch into desired shape and size and packing to make finished products.

In some embodiments, optionally in combination with any or all of above various embodiments, the composition of the present invention, is applied to the mammal subject by topical route, subcutaneous route, transdermal route, transcutaneous route, intradermal route, intramuscular route and percutaneous route.

In some embodiments, optionally in combination with any or all of above various embodiments, the composition of the present invention, is applied to the mammal subject using one or more of iontophoresis, microdialysis, ultrafiltration, electromagnetics, osmosis, electroosmosis, sonophoresis, electroporation, thermal poration, microporation, skin permeabilization or laser.

In some embodiments, optionally in combination with any or all of above various embodiments, the composition of the present invention, is applied to the mammal subject using a device. The device configured to deliver the composition of the present invention can include one or more of a microneedle, a microfine lance, a microfine cannula, a microinjector, a jet fluid injector and a high pressure jet fluid injector. The device configured to deliver the composition of the present invention can include one or more of topical delivery devices, transdermal delivery devices, transcutaneous delivery devices, percutaneous delivery devices, intradermal delivery devices, intramuscular devices and subcutaneous devices.

In some embodiments, optionally in combination with any or all of above various embodiments, the composition of the present invention, is topically applied to the specified area in liquid form. In some embodiments, the composition of the present invention is topically applied to the specified area by drops or a spray mechanism.

In some embodiments, optionally in combination with any or all of above various embodiments, the composition of the present invention, is topically applied to the specified areas of the mammal subject by applying the patches that contain the composition. In some embodiments, the composition of the present invention, is topically applied to the specified areas of the mammal subject by applying the gels, or pastes, or creams, or ointments, or lotions that all contain the composition.

### EXAMPLES

Although several embodiments have been described in detail for purposes of illustration, various modification may be made without departing from the scope and spirit of the invention. Accordingly, the invention is not to be limited, except as by the appended claims.

The method described herein are not limited to any particular targeting agent and a variety of targeting agents can be used.

Although the compositions in the examples below are formulated as a patch or liquid, the compositions employed in the subject methods and the composition of the present invention can be formulated as gels, pastes, creams, ointments and lotions.

### Example 1

### Liquid Formulation Containing Eugenol

TABLE 1

| | Percent by |
|---|---|
| Ingredients | Weight |
| Eugenol | 0.2-50.0 |
| Ethanol | 50.0-99.8 |
| Total | 100 |

Eugenol was added to ethanol and mixed until solubilized. The liquids may be directly applied onto the specified skin areas.

### Example 2

### Preparation of Patches Containing Eugenol

TABLE 2

| | Percent by |
|---|---|
| Ingredients | Weight |
| Polyacrylic acid | 3.0 |
| Sodium polyacrylate | 5.0 |
| Polyvinyl alcohol | 1.0 |
| Sodium carboxymethyl cellulose | 5.0 |
| Glycerin | 15.0 |
| Water | 45.0 |
| Aluminum hydroxide | 0.3 |
| Polysorbate 80 | 0.1 |
| Disodium edetate | 0.1 |
| Tartaric acid | 0.2 |
| Polyoxyethylene monostearate | 0.3 |
| Active ingredient concentrate | 25.0 |
| Total | 100 |

The ingredients shown in Table 2 in which the active ingredient concentrate is the liquid solutions from Example 1, were uniformly mixed to obtain the coating dispersions. The functions of the ingredients in Table 2 (except the active ingredient concentrate) are well known in patch manufacturing and an ordinary skilled artisan should be well understood.

Next, the coating dispersions were spread onto a polyethylene terephthalate (PET) nonwoven sheet to a coating weight of 500 g/m² and a polyethylene film was put on it. The product was cut into a fixed size and then sealed into a bag made of a packaging material that includes a layer of aluminum.

It should be noted that the above manufacturing protocol is merely illustrative. Any protocol that is capable of producing the subject patch preparations may be employed.

### Example 3

### Liquid Formulation Containing Camphor

TABLE 3

| | Percent by |
|---|---|
| Ingredients | Weight |
| Camphor | 0.5-50.0 |
| Ethanol | 50.0-99.5 |
| Total | 100 |

Camphor was added to ethanol and mixed until solubilized. The liquids may be directly applied onto the specified skin areas.

### Example 4

### Preparation of Patches Containing Camphor

The ingredients shown in Table 2 in which the active ingredient concentrate is the liquid solutions from Example 3, uniformly mixed to obtain the coating dispersions. The functions of the ingredients in Table 2 (except the active ingredient concentrate) are well known in patch manufacturing and an ordinary skilled artisan should be well understood.

Next, the coating dispersions were spread onto a polyethylene terephthalate (PET) nonwoven sheet to a coating weight of 500 g/m² and a polyethylene film was put on it. The product was cut into a fixed size and then sealed into a bag made of a packaging material that includes a layer of aluminum.

It should be noted that the above manufacturing protocol is merely illustrative. Any protocol that is capable of producing the subject patch preparations may be employed.

### Example 5

### Liquid Formulation Containing Borneol

TABLE 4

| | Percent by |
|---|---|
| Ingredients | Weight |
| Borneol | 0.02-50.0 |
| Ethanol | 50.0-99.98 |
| Total | 100 |

Borneol was added to ethanol and mixed until solubilized. The liquids may be directly applied onto the specified skin areas.

### Example 6

### Preparation of Patches Containing Borneol

The ingredients shown in Table 2 in which the active ingredient concentrate is the liquid solutions from Example 5, uniformly mixed to obtain the coating dispersions. The functions of the ingredients in Table 2 (except the active ingredient concentrate) are well known in patch manufacturing and an ordinary skilled artisan should be well understood.

Next, the coating dispersions were spread onto a polyethylene terephthalate (PET) nonwoven sheet to a coating weight of 500 g/m² and a polyethylene film was put on it. The product was cut into a fixed size and then sealed into a bag made of a packaging material that includes a layer of aluminum.

It should be noted that the above manufacturing protocol is merely illustrative. Any protocol that is capable of producing the subject patch preparations may be employed.

### Example 7

### Liquid Formulation Containing 1,8-Cineole

TABLE 5

| | Percent by |
|---|---|
| Ingredients | Weight |
| 1,8-Cineole | 0.2-50.0 |
| Solvent | 50.0-99.8 |
| Total | 100 |

The solvent consisted of 6.0% by weight glycerol tripalmitate, 3.0% by weight glycerol tristearate, 28.0% by weight triolein, 60.0% by weight trilinolein and 3.0% by weight trilinolenin. 1,8-Cineole was added to the solvent and mixed until solubilized. The liquids may be directly applied onto the specified skin areas.

### Example 8

### Preparation of Patches Containing 1,8-Cineole

The ingredients shown in Table 2 in which the active ingredient concentrate is the liquid solutions from Example 7, uniformly mixed to obtain the coating dispersions. The functions of the ingredients in Table 2 (except the active ingredient concentrate) are well known in patch manufacturing and an ordinary skilled artisan should be well understood.

Next, the coating dispersions were spread onto a polyethylene terephthalate (PET) nonwoven sheet to a coating weight of 500 g/m² and a polyethylene film was put on it. The product was cut into a fixed size and then sealed into a bag made of a packaging material that includes a layer of aluminum.

It should be noted that the above manufacturing protocol is merely illustrative. Any protocol that is capable of producing the subject patch preparations may be employed.

### Example 9

### Liquid Formulation Containing 6-Gingerol

TABLE 6

| | Percent by |
|---|---|
| Ingredients | Weight |
| 6-Gingerol | 0.2-20.0 |
| Solvent | 80.0-99.8 |
| Total | 100 |

The solvent consisted of 6.0% by weight glycerol tripalmitate, 3.0% by weight glycerol tristearate, 28.0% by weight triolein, 60.0% by weight trilinolein and 3.0% by weight trilinolenin. 6-Gingerol was added to the solvent and mixed until solubilized. The liquids may be directly applied onto the specified skin areas.

### Example 10

### Preparation of Patches Containing 6-Gingerol

The ingredients shown in Table 2 in which the active ingredient concentrate is the liquid solutions from Example 9, uniformly mixed to obtain the coating dispersions. The functions of the ingredients in Table 2 (except the active ingredient concentrate) are well known in patch manufacturing and an ordinary skilled artisan should be well understood.

Next, the coating dispersions were spread onto a polyethylene terephthalate (PET) nonwoven sheet to a coating weight of 500 g/m² and a polyethylene film was put on it. The product was cut into a fixed size and then sealed into a bag made of a packaging material that includes a layer of aluminum.

It should be noted that the above manufacturing protocol is merely illustrative. Any protocol that is capable of producing the subject patch preparations may be employed.

### Example 11

### Liquid Formulation Containing Citral

TABLE 7

| | Percent by |
|---|---|
| Ingredients | Weight |
| Citral | 0.2-50.0 |
| Solvent | 50.0-99.8 |
| Total | 100 |

The solvent consisted of 6.0% by weight glycerol tripalmitate, 3.0% by weight glycerol tristearate, 28.0% by weight triolein, 60.0% by weight trilinolein and 3.0% by weight trilinolenin. Citral was added to the solvent and mixed until solubilized. The liquids may be directly applied onto the specified skin areas.

### Example 12

### Preparation of Patches Containing Citral

The ingredients shown in Table 2 in which the active ingredient concentrate is the liquid solutions from Example 11, uniformly mixed to obtain the coating dispersions. The functions of the ingredients in Table 2 (except the active ingredient concentrate) are well known in patch manufacturing and an ordinary skilled artisan should be well understood.

Next, the coating dispersions were spread onto a polyethylene terephthalate (PET) nonwoven sheet to a coating weight of 500 g/m² and a polyethylene film was put on it. The product was cut into a fixed size and then sealed into a bag made of a packaging material that includes a layer of aluminum.

It should be noted that the above manufacturing protocol is merely illustrative. Any protocol that is capable of producing the subject patch preparations may be employed.

### Example 13

### Liquid Formulation Containing Citrate

TABLE 8

| | Percent by |
|---|---|
| Ingredients | Weight |
| Trisodium Citrate | 0.002-10.0 |
| Water | 50.0-99.8 |
| Total | 100 |

Trisodium citrate was added to water and mixed until solubilized. The liquids may be directly applied onto the specified skin areas.

### Example 14

### Preparation of Patches Containing Citrate

The ingredients shown in Table 9 in which the active ingredient concentrate is the liquid solutions from Example 13, uniformly mixed to obtain the coating dispersions. The functions of the ingredients in Table 9 (except the active ingredient concentrate) are well known in patch manufacturing and an ordinary skilled artisan should be well understood.

Next, the coating dispersions were spread onto a polyethylene terephthalate (PET) nonwoven sheet to a coating weight of 500 g/m² and a polyethylene film was put on it. The product was cut into a fixed size and then sealed into a bag made of a packaging material that includes a layer of aluminum.

It should be noted that the above manufacturing protocol is merely illustrative. Any protocol that is capable of producing the subject patch preparations may be employed.

TABLE 9

| | Percent by |
|---|---|
| Ingredients | Weight |
| Polyacrylic acid | 3.0 |
| Sodium polyacrylate | 5.0 |
| Polyvinyl alcohol | 1.0 |
| Sodium carboxymethyl cellulose | 5.0 |
| Glycerin | 20.0 |
| Water | 50.0 |
| Aluminum hydroxide | 0.3 |
| Polysorbate 80 | 0.1 |
| Disodium edetate | 0.1 |
| Tartaric acid | 0.2 |
| Polyoxyethylene monostearate | 0.3 |
| Active ingredient concentrate | 15.0 |
| Total | 100 |

### Example 15

### Liquid Formulation Containing Bisandrographolide A

TABLE 10

| | Percent by |
|---|---|
| Ingredients | Weight |
| Bisandrographolide A | 0.1-1.0 |
| Ethanol | 99.0-99.9 |
| Total | 100 |

Bisandrographolide A was added to ethanol and mixed until solubilized. The liquids may be directly applied onto the specified skin areas.

### Example 16

### Preparation of Patches Containing Bisandrographolide A

The ingredients shown in Table 2 in which the active ingredient concentrate is the liquid solutions from Example 15, uniformly mixed to obtain the coating dispersions. The functions of the ingredients in Table 2 (except the active ingredient concentrate) are well known in patch manufacturing and an ordinary skilled artisan should be well understood.

Next, the coating dispersions were spread onto a polyethylene terephthalate (PET) nonwoven sheet to a coating weight of 500 g/m² and a polyethylene film was put on it. The product was cut into a fixed size and then sealed into a bag made of a packaging material that includes a layer of aluminum.

It should be noted that the above manufacturing protocol is merely illustrative. Any protocol that is capable of producing the subject patch preparations may be employed.

### Example 17

### Liquid Formulation Containing 2-Aminoethoxydiphenyl Borate

TABLE 11

| | Percent by |
|---|---|
| Ingredients | Weight |
| 2-Aminoethoxydiphenyl borate | 0.2-2.2 |
| Dimethyl sulfoxide | 97.8-99.8 |
| Total | 100 |

2-Aminoethoxydiphenyl borate was added to dimethyl sulfoxide and mixed until solubilized. The liquids may be directly applied onto the specified skin areas.

### Example 18

### Preparation of Patches Containing Camphor

The ingredients shown in Table 2 in which the active ingredient concentrate is the liquid solutions from Example 18, uniformly mixed to obtain the coating dispersions. The functions of the ingredients in Table 2 (except the active ingredient concentrate) are well known in patch manufacturing and an ordinary skilled artisan should be well understood.

Next, the coating dispersions were spread onto a polyethylene terephthalate (PET) nonwoven sheet to a coating weight of 500 g/m² and a polyethylene film was put on it. The product was cut into a fixed size and then sealed into a bag made of a packaging material that includes a layer of aluminum.

It should be noted that the above manufacturing protocol is merely illustrative. Any protocol that is capable of producing the subject patch preparations may be employed.

### Example 19

### Liquid Formulation Containing Camphor and 1,8-Cineole

TABLE 12

| | Percent by |
|---|---|
| Ingredients | Weight |
| Camphor | 0.25-25 |
| 1,8-Cineole | 0.1-25.0 |
| Solvent | to 100.0 |
| Total | 100 |

The solvent consisted of 6.0% by weight glycerol tripalmitate, 3.0% by weight glycerol tristearate, 28.0% by weight triolein, 60.0% by weight trilinolein and 3.0% by weight trilinolenin. Camphor and 1,8-Cineole were added to the solvent and mixed until solubilized. The liquids may be directly applied onto the specified skin areas.

### Example 20

### Preparation of Patches Containing Camphor and 1,8-Cineole

The ingredients shown in Table 2 in which the active ingredient concentrate is the liquid solutions from Example 19, uniformly mixed to obtain the coating dispersions. The functions of the ingredients in Table 2 (except the active ingredient concentrate) are well known in patch manufacturing and an ordinary skilled artisan should be well understood.

Next, the coating dispersions were spread onto a polyethylene terephthalate (PET) nonwoven sheet to a coating weight of 500 g/m² and a polyethylene film was put on it. The product was cut into a fixed size and then sealed into a bag made of a packaging material that includes a layer of aluminum.

It should be noted that the above manufacturing protocol is merely illustrative. Any protocol that is capable of producing the subject patch preparations may be employed.

### Example 21

### Liquid Formulation Containing Borneol, Camphor and 1,8-Cineole

TABLE 13

| | Percent by |
|---|---|
| Ingredients | Weight |
| Borneol | 8.0 |
| Camphor | 0.25-25 |
| 1,8-Cineole | 0.1-25.0 |
| Solvent | to 100.0 |
| Total | 100 |

The solvent consisted of 6.0% by weight glycerol tripalmitate, 3.0% by weight glycerol tristearate, 28.0% by weight triolein, 60.0% by weight trilinolein and 3.0% by weight trilinolenin. Borneol, camphor and 1,8-cineole were added to the solvent and mixed until solubilized. The liquids may be directly applied onto the specified skin areas.

### Example 22

### Preparation of Patches Containing Borneol, Camphor and 1,8-Cineole

The ingredients shown in Table 2 in which the active ingredient concentrate is the liquid solutions from Example 21, uniformly mixed to obtain the coating dispersions. The functions of the ingredients in Table 2 (except the active ingredient concentrate) are well known in patch manufacturing and an ordinary skilled artisan should be well understood.

Next, the coating dispersions were spread onto a polyethylene terephthalate (PET) nonwoven sheet to a coating weight of 500 g/m² and a polyethylene film was put on it. The product was cut into a fixed size and then sealed into a bag made of a packaging material that includes a layer of aluminum.

It should be noted that the above manufacturing protocol is merely illustrative. Any protocol that is capable of producing the subject patch preparations may be employed.

### Example 23

### Liquid Formulation Containing Borneol, Camphor, 1,8-Cineole and Citral

The solvent consisted of 6.0% by weight glycerol tripalmitate, 3.0% by weight glycerol tristearate, 28.0% by weight triolein, 60.0% by weight trilinolein and 3.0% by weight trilinolenin. Borneol, camphor, 1,8-cineole and citral were added to the solvent and mixed until solubilized. The liquids may be directly applied onto the specified skin areas.

TABLE 14

| | Percent by |
|---|---|
| Ingredients | Weight |
| Borneol | 8.0 |
| Camphor | 0.25-25 |
| 1,8-Cineole | 0.1-25.0 |
| Citral | 5.0 |
| Solvent | to 100.0 |
| Total | 100 |

### Example 24

### Preparation of Patches Containing Borneol, Camphor, 1,8-Cineole and Citral

The ingredients shown in Table 2 in which the active ingredient concentrate is the liquid solutions from Example 23, uniformly mixed to obtain the coating dispersions. The functions of the ingredients in Table 2 (except the active ingredient concentrate) are well known in patch manufacturing and an ordinary skilled artisan should be well understood.

Next, the coating dispersions were spread onto a polyethylene terephthalate (PET) nonwoven sheet to a coating weight of 500 g/m² and a polyethylene film was put on it. The product was cut into a fixed size and then sealed into a bag made of a packaging material that includes a layer of aluminum.

It should be noted that the above manufacturing protocol is merely illustrative. Any protocol that is capable of producing the subject patch preparations may be employed.

### Example 25

### Liquid Formulation Containing Borneol, Camphor, 1,8-Cineole, Citral and Eugenol

The solvent consisted of 6.0% by weight glycerol tripalmitate, 3.0% by weight glycerol tristearate, 28.0% by weight triolein, 60.0% by weight trilinolein and 3.0% by weight trilinolenin. Borneol, camphor, 1,8-cineole, citral and eugenol were added to the solvent and mixed until solubilized. The liquids may be directly applied onto the specified skin areas.

TABLE 15

| | Percent by |
|---|---|
| Ingredients | Weight |
| Borneol | 8.0 |
| Camphor | 0.25-25 |
| 1,8-Cineole | 0.1-25.0 |
| Citral | 5.0 |
| Eugenol | 2.0 |
| Solvent | to 100.0 |
| Total | 100 |

### Example 26

### Preparation of Patches Containing Borneol, Camphor, 1,8-Cineole, Citral and Eugenol

The ingredients shown in Table 2 in which the active ingredient concentrate is the liquid solutions from Example 25, uniformly mixed to obtain the coating dispersions. The functions of the ingredients in Table 2 (except the active ingredient concentrate) are well known in patch manufacturing and an ordinary skilled artisan should be well understood.

Next, the coating dispersions were spread onto a polyethylene terephthalate (PET) nonwoven sheet to a coating weight of 500 g/m² and a polyethylene film was put on it. The product was cut into a fixed size and then sealed into a bag made of a packaging material that includes a layer of aluminum.

It should be noted that the above manufacturing protocol is merely illustrative. Any protocol that is capable of producing the subject patch preparations may be employed.

### Example 27

### Liquid Formulation Containing Eugenol and Bisandrographolide A

TABLE 16

| | Percent by |
|---|---|
| Ingredients | Weight |
| Eugenol | 0.2-10.0 |
| Bisandrographolide A | 0.6 |
| Ethanol | 89.4-99.2 |
| Total | 100 |

Eugenol and bisandrographolide A were added to ethanol and mixed until solubilized. The liquids may be directly applied onto the specified skin areas.

### Example 28

### Preparation of Patches Containing Eugenol and Bisandrographolide A

The ingredients shown in Table 2 in which the active ingredient concentrate is the liquid solutions from Example 27, uniformly mixed to obtain the coating dispersions. The functions of the ingredients in Table 2 (except the active ingredient concentrate) are well known in patch manufacturing and an ordinary skilled artisan should be well understood.

Next, the coating dispersions were spread onto a polyethylene terephthalate (PET) nonwoven sheet to a coating weight of 500 g/m² and a polyethylene film was put on it. The product was cut into a fixed size and then sealed into a bag made of a packaging material that includes a layer of aluminum.

It should be noted that the above manufacturing protocol is merely illustrative. Any protocol that is capable of producing the subject patch preparations may be employed.

### Example 29

### Preparation of Patches Containing Borneol, Camphor, 1,8-Cineole, Citral, Eugenol and Bisandrographolide A

The ingredients shown in Table 17, in which the active ingredient concentrate 1 is the liquid solutions from Example 27 and the active ingredient concentrate 2 is the bisandrographolide A solution 0.6% by weight in ethanol, were uniformly mixed to obtain the coating dispersions. The functions of the ingredients in Table 17 (except the active ingredient concentrate1 and active ingredient concentrate 2) are well known in patch manufacturing and an ordinary skilled artisan should be well understood.

TABLE 17

| | Percent by |
|---|---|
| Ingredients | Weight |
| Polyacrylic acid | 3.0 |
| Sodium polyacrylate | 5.0 |
| Polyvinyl alcohol | 1.0 |
| Sodium carboxymethyl cellulose | 5.0 |
| Glycerin | 15.0 |
| Water | 45.0 |
| Aluminum hydroxide | 0.3 |
| Polysorbate 80 | 0.1 |
| Disodium edetate | 0.1 |
| Tartaric acid | 0.2 |
| Polyoxyethylene monostearate | 0.3 |
| Active ingredient concentrate 1 | 12.5 |
| Active ingredient concentrate 2 | 12.5 |
| Total | 100 |

Next, the coating dispersions were spread onto a polyethylene terephthalate (PET) nonwoven sheet to a coating weight of 500 g/m² and a polyethylene film was put on it. The product was cut into a fixed size and then sealed into a bag made of a packaging material that includes a layer of aluminum.

It should be noted that the above manufacturing protocol is merely illustrative. Any protocol that is capable of producing the subject patch preparations may be employed.

### Example 30

### Liquid Formulation Containing Borneol, Camphor, 1,8-Cineole, Terpinen-4-ol and α-Terpineol

The solvent consisted of 6.0% by weight glycerol tripalmitate, 3.0% by weight glycerol tristearate, 28.0% by weight triolein, 60.0% by weight trilinolein and 3.0% by weight trilinolenin. The ingredients shown in Table 18 were uniformly mixed to obtain the liquids. The liquids may be directly applied onto the specified skin areas.

TABLE 18

| | Percent by |
|---|---|
| Ingredients | Weight |
| Borneol | 2.0 |
| Camphor | 0.25-25 |
| 1,8-Cineole | 0.1-25.0 |
| Terpinen-4-ol | 1.0 |
| α-Terpineol | 6.0 |
| Solvent | to 100.0 |
| Total | 100 |

### Example 31

### Preparation of Patches Containing Borneol, Camphor, 1,8-Cineole, Terpinen-4-ol and α-Terpineol

The ingredients shown in Table 2 in which the active ingredient concentrate is the liquid solutions from Example 30, uniformly mixed to obtain the coating dispersions. The functions of the ingredients in Table 2 (except the active ingredient concentrate) are well known in patch manufacturing and an ordinary skilled artisan should be well understood.

Next, the coating dispersions were spread onto a polyethylene terephthalate (PET) nonwoven sheet to a coating weight of 500 g/m² and a polyethylene film was put on it. The product was cut into a fixed size and then sealed into a bag made of a packaging material that includes a layer of aluminum.

It should be noted that the above manufacturing protocol is merely illustrative. Any protocol that is capable of producing the subject patch preparations may be employed.

### Example 32

### Liquid Formulation Containing Borneol, Camphor, 1,8-Cineole, Trans-Caryophyllene and Thujone

TABLE 19

| | Percent by |
|---|---|
| Ingredients | Weight |
| Borneol | 2.0 |
| Camphor | 0.25-25 |
| 1,8-Cineole | 0.1-25.0 |
| trans-Caryophyllene | 3.0 |
| Thujone | 5.0 |
| Solvent | to 100.0 |
| Total | 100 |

The solvent consisted of 6.0% by weight glycerol tripalmitate, 3.0% by weight glycerol tristearate, 28.0% by weight triolein, 60.0% by weight trilinolein and 3.0% by weight trilinolenin. The ingredients shown in Table 19 uniformly mixed to obtain the liquids. The liquids may be directly applied onto the specified skin areas.

### Example 33

### Preparation of Patches Containing Borneol, Camphor, 1,8-Cineole, Trans-Caryophyllene and Thujone

The ingredients shown in Table 2 in which the active ingredient concentrate is the liquid solutions from Example 32, uniformly mixed to obtain the coating dispersions. The functions of the ingredients in Table 2 (except the active ingredient concentrate) are well known in patch manufacturing and an ordinary skilled artisan should be well understood.

Next, the coating dispersions were spread onto a polyethylene terephthalate (PET) nonwoven sheet to a coating weight of 500 g/m² and a polyethylene film was put on it. The product was cut into a fixed size and then sealed into a bag made of a packaging material that includes a layer of aluminum.

It should be noted that the above manufacturing protocol is merely illustrative. Any protocol that is capable of producing the subject patch preparations may be employed.

### Example 34

### Liquid Formulation Containing Borneol, Camphor, 1,8-Cineole, Terpinen-4-ol, α-Terpineol, Trans-Caryophyllene and Thujone

The solvent consisted of 6.0% by weight glycerol tripalmitate, 3.0% by weight glycerol tristearate, 28.0% by weight triolein, 60.0% by weight trilinolein and 3.0% by weight trilinolenin. The ingredients shown in Table 20 uniformly mixed to obtain the liquids. The liquids may be directly applied onto the specified skin areas.

TABLE 20

| | Percent by |
|---|---|
| Ingredients | Weight |
| Borneol | 2.0 |
| Camphor | 0.25-25 |
| 1,8-Cineole | 0.1-25.0 |
| Terpinen-4-ol | 1.0 |
| α-Terpineol | 6.0 |
| trans-Caryophyllene | 2.0 |
| Thujone | 5.0 |
| Solvent | to 100.0 |
| Total | 100 |

### Example 35

### Preparation of Patches Containing Borneol, Camphor, 1,8-Cineole, Terpinen-4-ol, α-Terpineol, Trans-Caryophyllene and Thujone

The ingredients shown in Table 2 in which the active ingredient concentrate is the liquid solutions from Example 34, uniformly mixed to obtain the coating dispersions. The functions of the ingredients in Table 2 (except the active ingredient concentrate) are well known in patch manufacturing and an ordinary skilled artisan should be well understood.

Next, the coating dispersions were spread onto a polyethylene terephthalate (PET) nonwoven sheet to a coating weight of 500 g/m² and a polyethylene film was put on it. The product was cut into a fixed size and then sealed into a bag made of a packaging material that includes a layer of aluminum.

It should be noted that the above manufacturing protocol is merely illustrative. Any protocol that is capable of producing the subject patch preparations may be employed.

### Example 36

### Preparation of a Patch Containing Borneol, Camphor, 1,8-Cineole, Terpinen-4-ol, α-Terpineol and Trans-Caryophyllene

### STEP 1

### The Preparation of a Liquid Solution of Selected Ingredients

The ingredients shown in Table 21, in which the solvent consisted of 6.0% by weight glycerol tripalmitate, 3.0% by weight glycerol tristearate, 28.0% by weight triolein, 60.0% by weight trilinolein and 3.0% by weight trilinolenin, were uniformly dissolved to obtain a liquid solution.

### STEP 2

### The Preparation of the Patch

The ingredients shown in Table 22 in which the active ingredient concentrate is the liquid solution from Step 1, uniformly mixed to obtain the coating dispersions. The functions of the ingredients in Table 22 (except the active ingredient concentrate) are well known in patch manufacturing and an ordinary skilled artisan should be well understood.

TABLE 21

| | Percent by |
|---|---|
| Ingredients | Weight |
| Borneol | 0.5 |
| Camphor | 7.6 |
| 1,8-Cineole | 2.5 |
| Terpinen-4-ol | 1.0 |
| α-Terpineol | 5.5 |
| trans-Caryophyllene | 1.0 |
| Solvent | to 100.0 |
| Total | 100 |

Next, the coating dispersions were spread onto a polyethylene terephthalate (PET) nonwoven sheet to a coating weight of 500 g/m² and a polyethylene film was put on it. The product was cut into a fixed size and then sealed into a bag made of a packaging material that includes a layer of aluminum.

It should be noted that the above manufacturing protocol is merely illustrative. Any protocol that is capable of producing the subject patch preparations may be employed.

TABLE 22

| | Percent by |
|---|---|
| Ingredients | Weight |
| Polyacrylic acid | 3.0 |
| Sodium polyacrylate | 5.0 |
| Polyvinyl alcohol | 1.0 |
| Sodium carboxymethyl cellulose | 5.0 |
| Glycerin | 20.0 |
| Water | 55.0 |
| Aluminum hydroxide | 0.3 |
| Polysorbate 80 | 0.1 |
| Disodium edetate | 0.1 |
| Tartaric acid | 0.2 |
| Polyoxyethylene monostearate | 0.3 |
| Active ingredient concentrate | 10.0 |
| Total | 100 |

### Example of Treatment

70 yr old female suffering from coronary artery disease (CAD) over the years and experiencing frequent vomiting and dizziness accompanying a headache. Applied the patches produced in Example 36 to the acupoints PC6 (Neiguan), LI4 (Hegu) and ST36 (Zusanli) over eight hours per day, resulting in complete relief from vomiting, dizziness and headache within one week.

What is claimed is:

1. A method of stimulating therapeutic points of a mammal subject, said method comprising applying to a set of therapeutic points or one or two or more therapeutic points of a mammal subject a therapeutically effective amount of a composition comprising an agonist of a transient receptor potential (TRP) channel selected from the group consisting of TRPV1, TRPV2, TRPV3, TRPV4, TRPA1, TRPM8, and combinations thereof.

2. The method as claimed in claim 1, wherein the said composition comprises one or two or more transient receptor potential (TRP) channel agonists selected from the group consisting of 2-aminoethoxydiphenol borate, borneol, bisandrographolide A, camphor, 1,8-cineole, citral, citrate, eugenol and 6-gingerol.

3. The method as claimed in claim 2, wherein 2-aminoethoxydiphenol borate, borneol, bisandrographolide A, camphor, 1,8-cineole, citral, citrate, eugenol and 6-gingerol are each between 0.001% and 50% by weight of total composition weight.

4. The method as claimed in claim 1, wherein the said composition further comprises one or two compounds selected from the group consisting of terpinen-4-ol and α-terpineol.

5. The method as claimed in claim 1, wherein the said composition further comprises one or two compounds selected from the group consisting of trans-caryophyllene and thujone.

6. The method as claimed in claim 1, wherein the said composition is applied topically as a patch.

7. A method of treating a disease of a mammal subject, comprising:
i. choosing a set of therapeutic points or one or two or more therapeutic points of the mammal subject according to subject's symptoms;
ii. applying to the chosen therapeutic point(s) of the subject a therapeutically effective amount of a composition comprising an agonist of a transient receptor potential (TRP) channel selected from the group consisting of TRPV1, TRPV2, TRPV3, TRPV4, TRPA1, TRPM8, and combinations thereof.

8. The method as claimed in claim 7, wherein the said composition comprises one or two or more transient receptor potential (TRP) channel agonists selected from the group consisting of 2-aminoethoxydiphenol borate, borneol, bisandrographolide A, camphor, 1,8-cineole, citral, citrate, eugenol and 6-gingerol.

9. The method as claimed in claim 8, wherein 2-aminoethoxydiphenol borate, borneol, bisandrographolide A, camphor, 1,8-cineole, citral, citrate, eugenol and 6-gingerol are each between 0.001% and 50% by weight of total composition weight.

10. The method as claimed in claim 7, wherein the said composition further comprises one or two compounds selected from the group consisting of terpinen-4-ol and α-terpineol.

11. The method as claimed in claim 7, wherein the said composition further comprises one or two compounds selected from the group consisting of trans-caryophyllene and thujone.

12. The method as claimed in claim 7, wherein the said composition is applied topically as a patch.

13. The method as claimed in claim 7, wherein the said therapeutic point comprises one or two or more therapeutic points selected from the group consisting of LU7 (Lieque), LU10 (Yuji), LU11 (Shaoshang), LI1 (Shangyang), LI4 (Hegu), LI11 (Quchi), LI20 (Yingxiang), ST6 (Jiache), ST7 (Xiaguan), ST25 (Tianshu), ST34 (Liangqiu), ST36 (Zusanli), ST37 (Shangjuxu), ST39 (Xiajuxu), ST40 (Fenglong), ST44 (Neiting), SP4 (Gongsun), SP6 (Sanyinjiao), SP8 (Diji), SP9 (Yinlingquan), SP10 (Xuehai), SP21 (Dabao), HT5 (Tongli), HT6 (Yinxi), HT9 (Shaochong), SI1 (Shaoze), S13 (Houxi), S119 (Tinggong), BL2 (Cuanzhu), BL7 (Tongtian), BL10 (Tianzhu), BL13 (Feishu), BL15 (Xinshu), BL17 (Geshu), BL21 (Weishu), BL23 (Shenshu), BL60 (Kunlun), KI1 (Yongquan), K13 (Taixi), K15 (Shuiquan), K16 (Zhaohai), K17 (Fuliu), PC4 (Ximen), PC6 (Neiguan), PC7 (Daling), PC8 (Laogong), PC9 (Zhongchong), TE1 (Guanchong), TE3 (Zhongzhu), TE5 (Waiguan), TE6 (Zhigou), TE17 (Yifeng), TE21 (Ermen), TE23 (Sizhukong), GB2 (Tinghui), GB20 (Fengchi), GB24 (Riyue), GB25 (Jingmen), GB31 (Fengshi), GB34 (Yanglingquan), GB39 (Xuanzhong), GB41 (Zulinqi), LR3 (Taichong), LR13 (Zhangmen), GV1 (Changqiang), GV8 (Jinsuo), GV9 (Zhiyang), GV12 (Shenzhu), GV14 (Dazhui), GV15 (Yamen), GV20 (Baihui), GV23 (Shangxing), GV26 (Shuigou), CV3 (Zhongji), CV4 (Guanyuan), CV6 (Qihai), CV8 (Shenque), CV12 (Zhongwan), CV17 (Danzhong), CV22 (Tiantu), CV23 (Lianquan), CV24 (Chengjiang), EX-HN3 (Yintang), EX-HN5 (Taiyang), EX-B1 (Dingchuan), EX-UE11 (Shixuan) and EX-LE6 (Dannang).

14. The method as claimed in claim 7, wherein the disease is selected from the group consisting of fever, convulsions, diarrhea, constipation, coma, rectal prolapse, prostration, enuresis, cough, anuresis, asthma, renal colic, excessive phlegm, dysmenorrhea, hyperhidrosis, chest tightness, chest pain, night sweats, hypochondriac pain, dizziness, stiff neck, insomnia, excessive dreaming, pruritus, palpitations, eye pain, swollen eyes, cardialgia, nasal congestion, rhinorrhea, stomach pain, tinnitus, hearing loss, nausea, vomiting, halitosis, hiccups, toothache, jaundice, trismus, biliary colic, sore throat, abdominal distension, abdominal pain and aphasia.

15. A composition which stimulates a set of therapeutic points or one or two or more therapeutic points of a mammal subject, comprising one or more transient receptor potential (TRP) channel agonists selected from the group consisting of 2-aminoethoxydiphenol borate, borneol, bisandrographolide A, camphor, 1,8-cineole, citral, citrate, eugenol and 6-gingerol.

16. The composition as claimed in claim 15, wherein 2-aminoethoxydiphenol borate, borneol, bisandrographolide A, camphor, 1,8-cineole, citral, citrate, eugenol and 6-gingerol are each between 0.001% and 50% by weight of total composition weight.

17. The composition as claimed in claim 15 further comprises one or two compounds selected from the group consisting of terpinen-4-ol and α-terpineol.

18. The composition as claimed in claim 15 further comprises one or two compounds selected from the group consisting of trans-caryophyllene and thujone.

19. The composition as claimed in claim 15, wherein it is applied topically as a patch.

20. The composition as claimed in claim 15, wherein it is applied topically in liquid form.
